SEQUENCE LISTING (17 sequences)

SEQ ID NO: 1 - protein, 136 aa, Homo sapiens
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 2 - protein, 136 aa, Homo sapiens
MARTKQTARKSTGGKAPRKQLATKAARKSAPATGGVKKPHRYRPGTVALREIRRYQKSTELLIRKLPFQRLVREIAQDFKTDLRFQSSAVMALQEACEAYLVGLFEDTNLCAIHAKRVTIMPKDIQLARRIRGERA

SEQ ID NO: 3 - protein, 136 aa, Homo sapiens
MARTKQTARKSTGGKAPRKQLATKAARMSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 4 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

SEQ ID NO: 5 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 6 - DNA, 20 bp, Artificial sequence
  Primer
cacactctgctcctgacctg

SEQ ID NO: 7 - DNA, 20 bp, Artificial sequence
  Primer
gcagtgttttcaggggtgtt

SEQ ID NO: 8 - DNA, 20 bp, Artificial sequence
  Primer
gcagcagcaacaacaaaaac

SEQ ID NO: 9 - DNA, 20 bp, Artificial sequence
  Primer
ccatctgcagtcgggtaact

SEQ ID NO: 10 - DNA, 19 bp, Artificial sequence
  Primer
ggaacgtccctctgcctag

SEQ ID NO: 11 - DNA, 20 bp, Artificial sequence
  Primer
cagacagtagctcccatgcc

SEQ ID NO: 12 - DNA, 20 bp, Artificial sequence
  Primer
cattgtgaaaaggggtcgtt

SEQ ID NO: 13 - DNA, 20 bp, Artificial sequence
  Primer
tgaaagccaagaggagagga

SEQ ID NO: 14 - DNA, 21 bp, Artificial sequence
  Primer
acagtcaagtgctgcaaaaga

SEQ ID NO: 15 - DNA, 20 bp, Artificial sequence
  Primer
tccaacctcaacactgtcct

SEQ ID NO: 16 - DNA, 20 bp, Artificial sequence
  Primer
acatcctcattgagttgcgc

SEQ ID NO: 17 - DNA, 20 bp, Artificial sequence
  Primer
cagctcggagtctaggtcag

(19) United States
(12) Patent Application Publication
(10) Pub. No.: US 2018/0271857 A1
(43) Pub. Date: Sep. 27, 2018

(54) TREATMENT OF CANCER BY INHIBITING EZH2 ACTIVITY

(71) Applicant: University of Copenhagen, Copenhagen K (DK)

(72) Inventors: Kristian HELIN, Charlottenlund (DK); Faizaan MOHAMMAD, Frederiksberg (DK); Jonas Westergaard HØJFELDT, Birkerød (DK); Deo Prakash PANDEY, Copenhagen (DK); Simon WEISSMANN, Copenhagen Ø (DK)

(73) Assignee: University of Copenhagen, Copenhagen K (DK)

(21) Appl. No.: 15/538,434

(22) PCT Filed: Dec. 18, 2015
(86) PCT No.: PCT/DK2015/050407

§ 371 Date: Jun. 21, 2017

(30) Foreign Application Priority Data
Dec. 23, 2014 (DK) PA 2014 70826

(51) Int. Cl. A61K 31/496; A61K 31/5377; C12Q 1/6886; A61P 35/00

(52) U.S. Cl. CPC A61K 31/496; A61K 31/5377; C12Q 2600/106; A61P 35/00; C12Q 1/6886

(57) ABSTRACT

The invention relates to inhibitors of EZH2 for use in the treatment of cancers characterised by expression of mutated histone H3 having a mutation of amino acid number 27. The invention also relates to methods for predicting the efficacy of treatment of a cancer with an inhibitor of EZH2 by determining whether the cancer cells contain a gene encoding p16^{INK4A}, wherein the presence of a gene encoding p16^{INK4A} is indicative of efficacy of treatment of the cancer with an inhibitor of EZH2.

### FIELD OF INVENTION

The present invention relates to methods for treatment of cancer using inhibitors of EZH2.

### BACKGROUND OF INVENTION

Trimethylation of K27 on histone H3 (H3K27me3) is a repressive epigenetic modification that is catalyzed by polycomb repressive complex 2 (PRC2) of which EZH2 is the catalytic subunit. PRC2 is required for normal embryonic development and differentiation.

The role of EZH2 in cancer remains elusive. Thus as discussed by Hanno Hock, 2012, then on the one hand disruption of Ezh2 in mice is sufficient to cause T-acute lymphoblastic leukemia (T-ALL), and similar mechanisms are involved in human T-ALL. On the other hand EZH2 activity may promote cancer and its activity is frequently deregulated in cancer. EZH2 is amplified and/or over-expressed in variety of solid tumors including prostate, kidney, breast and colorectal cancer and often the elevated EZH2 activity in tumors is associated with poor prognosis. In addition, somatic activating point mutations EZH2 have been identified in non-Hodgkin lymphoma suggesting that increased PRC2 activity is a recurrent event in cancers and this has given impetus to develop EZH2 inhibitors as potential anti-cancer drugs.

Recent discovery of mutations in histone H3 genes in pediatric glioma has revealed another approach through which cancer cells can modulate PRC2 activity. Diffuse intrinsic pontine glioma (DIPG) is the most aggressive primary brain tumor that originates in pons and found exclusively in children. Up to 88% of DIPG tumors show a p.Lys27Met (K27M) mutation in H3 genes including genes encoding canonical H3.3 (H3F3A) and variant H3.1 (HIST1H3B) histones. Histone H3 with K27M mutation (H3K27M) has been shown to inhibit PRC2 activity in vitro by directly binding to EZH2 (Lewis et al., 2013). DIPG tumors with K27M mutation or cells expressing exogenous H3K27M show global loss of H3K27me3 level (Lewis et al., 2013). However, how H3K27M contributes to tumorigenesis is not well understood.

### SUMMARY OF INVENTION

There is thus a need for useful treatments of DIPG and other tumours characterised by a H3K27M mutation.

Surprisingly, the present invention discloses that inhibitors of EZH2 are very useful for treatment of cancers characterised by a H3K27M mutation.

This is highly surprising, because tumor cells expressing H3K27M are characterized by a global reduction of H3K27me3 levels, which is widely believed to be mechanistically important for tumorigenesis, but never-the-less such tumour cells are still sensitive to EZH2 inhibition. EZH2 inhibition leads to reduction of H3K37me3 levels, however in tumours characterised by a H3K27M mutation, these levels are already reduced.

Thus, it is an aspect of the present invention to provide an inhibitor of EZH2 for use in the treatment of cancer in an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

It is also an aspect of the invention to provide methods for predicting the efficacy of treatment of a cancer with an inhibitor of EZH2 in an individual in need thereof, said method comprising the steps of
i) providing a sample comprising cells of said cancer from said individual,
ii) determining whether said cells contain a gene encoding p16^{INK4A},

wherein the presence of a gene encoding p16^{INK4A} in said cells is indicative of efficacy of treatment of the cancer in said individual with an inhibitor of EZH2.

It is also an aspect of the invention to provide methods of treatment of cancer comprising administering a therapeutically effective amount of an inhibitor of EZH2 to an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

It is also an aspect of the invention to provide methods for treatment of cancer in an individual in need thereof, wherein the method comprises the steps of:
i) Obtaining information of whether cells of the cancer from said individual comprises a gene encoding p16^{INK4A}; and
ii) if said cancer cells contain a gene encoding p16^{INK4A}, then administering a therapeutically effective amount of said inhibitor of EZH2 to said individual,

thereby treating cancer in said individual.

It is also an aspect of the invention to provide use of an inhibitor of EZH2 for the preparation of a medicament for treatment of a cancer in an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

### DESCRIPTION OF DRAWINGS

FIG. 1 shows results of experiments with a DIPG mouse model. (A) Immunoblot showing exogenous expression of PDGFB together with WT (SEQ ID NO:1) or K27M mutated H3.3 (SEQ ID NO:3) in mouse NSCs. Immunoblot also shows the global loss of H3K27me3 and H3K27me2 levels in PDGFB/H3.3K27M NSCs. (B) Survival curve of SCID mice injected in pons with 10⁴ PDGFB NSCs expressing WT (SEQ ID NO:1) (n=4) (upper curve) or K27M mutated H3.3 (SEQ ID NO:3) (n=6) (lower curve). (C) Immunohistochemistry of brain of mouse injected with NSCs expressing PDGFB/H3.3 WT (SEQ ID NO:1) or PDGFB/H3.3K27M (SEQ ID NO:3) showing tumor localization in pons. The tumors showed strong staining for Nestin, a marker for undifferentiated neural stem cells and H3K27me3 staining is lacking in H3.3 K27M expressing tumor.

FIG. 2 shows that Ezh2 inhibition affects the growth of tumor cells in mouse DIPG model. (A) Immunoblots showing H3K27me3 and H3K27me2 levels in PDGFB NSCs treated with two different EZH2 inhibitors (GSK343 and EPZ6438) at different concentrations for 3 days. (B) In vitro cell proliferation assay of PDGFB NSCs expressing WT (SEQ ID NO:1) or K27M mutated H3.3 (SEQ ID NO:3) upon treatment with two different EZH2 inhibitors as indicated (3 μM). (C) Colony formation assay of DMSO or EZH2 inhibitor treated PDGFB NSCs expressing WT (SEQ ID NO:1) or K27M mutated H3.3 (SEQ ID NO:3). 2000 cells were plated and treated with DMSO, GSK343 or EPZ6438. Colonies formed after 9 days were fixed and stained with crystal violet. (D) Immunoblots showing the H3K27me3 and H3K27me2 levels as well as expression of p16^{Ink4a} in DMSO or EZH2 inhibitor treated (3 μM, 12 days) NSCs. (E) ChIP-qPCR analysis showing the enrichment of H3K27me3 over the Ink4a locus in DMSO or EZH2 inhibitor treated (3 μM, 12 days) PDGFB NSCs expressing WT or K27M mutated H3.3 as indicated on the right hand side of the figure (the order listed indicates the order of the columns shown in the left to right direction). Mouse Ink4a locus and the location of the primers used for the analysis is also shown. (F) H3K27me3 is strongly reduced on several EZH2 target genes in cells expressing H3K27M and by treatment with EPZ6438 as determined by ChIP-qPCR. Right hand list indicates the order of the columns shown in the left to right direction. (G) Tracks from ChIP-seq analysis showing H3K27me3 enrichment over Ink4a locus in DMSO or EZH2 inhibitor treated (3 μM, 12 days) PDGFB NSCs expressing WT (SEQ ID NO:1) or K27M mutated H3.3 (SEQ ID NO:3).

FIG. 3 shows that Ezh2 is required for growth of tumour cells in vivo.

(a) Immunoblot showing complete loss of Ezh2 after 8 days of 4-OHT treatment of Ezh2f/f; PDGFB/H3K27M NSCs. (b) Kaplan-Meier curve showing survival of mice in which 10⁵ Ezh2f/f; PDGFB/H3K27M NSCs pre-treated with ethanol (lower curve) or 4-OHT (upper curve) were injected into the pons. (c) Kaplan-Meier curve showing survival of mice in which 10⁴ Ezh2f/f; PDGFB/H3K27M NSCs were injected into the pons, and either treated with oil (n=5) (lower curve) or tamoxifen (n=5) (upper curve). The treatment periods are indicated as three bars.

FIG. 4 shows Effect of EZH2 inhibitors on adult GBM cells. Cell proliferation of DMSO or EZH2 inhibitor treated Ink4a/Arf−/−*EGFR NSCs.

### DETAILED DESCRIPTION OF THE INVENTION

Inhibitor of EZH2

The invention relates to methods for treatment of cancer involving use of an inhibitor of EZH2. The invention also relates to inhibitors of EZH2 for treatment of cancer. Said inhibitor of EZH2 may be any inhibitor of EZH2. In particular the inhibitor of EZH2 is a compound capable of reducing or completely inhibiting the activity of EZH2, wherein the activity of EZH2 is trimethylation of K27 in histone H3 (H3K27me3). Said histone H3 may for example be histone H3.3 of SEQ ID NO:1 or histone H3.1 or SEQ ID NO:2.

The term EZH2 as used herein refers to the protein EZH2. Ezh2 is the mammalian homolog of Enhancer of Zeste, the catalytic component of Polycomb repressive complex 2 (PRC2). The sequence of human EZH2 is provided herein as SEQ ID NO:4. Thus, the inhibitor of EZH2 may be an inhibitor of EZH2 of SEQ ID NO:4.

Since EZH2 in general is active within the polycomb repressive complex 2 (PRC2), said inhibitor of EZH2 may be a compound capable of reducing or even inhibiting catalysation of trimethylation of K27 on histone H3 (H3K27me3) by PRC2.

In general the inhibitor of EZH2 is a compound having an IC₅₀ with regard to inhibiting trimethylation of K27 on histone H3 (H3K27me3) by PRC2 or by EZH2 of <10 μM, more preferably <500 nM, even more preferably <50 nM.

The skilled person is well aware of useful methods for determining whether a compound is an inhibitor of EZH2. For example the assay described in Example 2 herein below may be used to determine whether a compound is an inhibitor of EZH2. It is preferred that the inhibitor of EZH2 has an IC₅₀<10 μM, more preferably the inhibitor of EZH2 has an IC₅₀<500 nM, even more preferably the inhibitor of EZH2 has an IC₅₀<50 nM when determined as described in Example 2.

In one embodiment of the invention the inhibitor of EZH2 is a compound comprising the core structure provided by formula D:

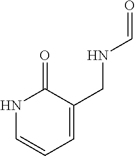 [SMILES: O=CNCc1ccc[nH]c1=O]

As used herein the term “compound comprising the core structure” means that the compound comprises the entire core structure. Thus, said compound may be the core structure substituted at one or more positions. By the term “substituted” in relation to organic compounds is meant that an —H is substituted by another moiety.

In one embodiment of the invention the inhibitor of EZH2 may be any of the inhibitors of EZH2 described in international patent application WO2011/140325. For example the inhibitor of EZH2 may be a compound of the formula (A)

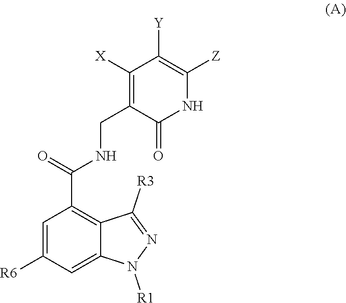

wherein

X and Z are selected independently from the group consisting of hydrogen, (C1-C8)alkyl, (C2-C8)alkenyl, (C2-C8)alkynyl, unsubstituted or substituted (C3-C8)cycloalkyl, unsubstituted or substituted (C3-C8)cycloalkyl-(C1-C8)alkyl or —(C2-C8)alkenyl, unsubstituted or substituted (C5-C8)cycloalkenyl, unsubstituted or substituted (C5-C8)cycloalkenyl-(C1-C8)alkyl or —(C2-C8)alkenyl, (C6-C10)bicycloalkyl, unsubstituted or substituted heterocycloalkyl, unsubstituted or substituted heterocycloalkyl-(C1-C8)alkyl or —(C2-C8)alkenyl, unsubstituted or substituted aryl, unsubstituted or substituted aryl-(C1-C8)alkyl or —(C2-C8)alkenyl, unsubstituted or substituted heteroaryl, unsubstituted or substituted heteroaryl-(C1-C8)alkyl or —(C2-C8)alkenyl, halo, cyano, —CORa, —CO2Ra, —CONRaRb, —CONRaNRaRb, —SRa, —SORa, —SO2Ra, —SO2NRaRb, nitro, —NRaRb, —NRaC(O)Rb, —NRaC(O)NRaRb, —NRaC(O)ORa, —NRaSO2Rb, —NRaSO2NRaRb, —NRaNRaRb, —NRaNRaC(O)Rb, —NRaNRaC(O)NRaRb, —NRaNRaC(O) ORa, —ORa, —OC(O)Ra, and —OC(O)NRaRb;

Y is H or halo;

R1 is (C1-C8)alkyl, (C2-C8)alkenyl, (C2-C8)alkynyl, unsubstituted or substituted (C3-C8)cycloalkyl, unsubstituted or substituted (C3-C8)cycloalkyl-(C1-C8)alkyl or —(C2-C8)alkenyl, unsubstituted or substituted (C5-C8)cycloalkenyl, unsubstituted or substituted (C5-C8)cycloalkenyl- (C1-C8)alkyl or —(C2-C8)alkenyl, unsubstituted or substituted (C6-C10)bicycloalkyl, unsubstituted or substituted heterocycloalkyl or —(C2-C8)alkenyl, unsubstituted or substituted heterocycloalkyl-(C1-C8)alkyl, unsubstituted or substituted aryl, unsubstituted or substituted aryl-(C1-C8)alkyl or —(C2-C8)alkenyl, unsubstituted or substituted heteroaryl, unsubstituted or substituted heteroaryl-(C1-C8)alkyl or —(C2-C8)alkenyl, —CORa, —CO2Ra, —CONRaRb, —CONR<a>NR<a>R<b>; R<3> is hydrogen, (C1-C8)alkyl, cyano, trifluoromethyl, —NR<a>R<b>, or halo; R<6> is selected from the group consisting of hydrogen, halo, (C1-C8)alkyl, (C2-C8)alkenyl, —B(OH)2, substituted or unsubstituted (C2-C8)alkynyl, unsubstituted or substituted (C3-C8)cycloalkyl, unsubstituted or substituted (C3-C8)cycloalkyl-(C1-C8)alkyl, unsubstituted or substituted (C5-C8)cycloalkenyl, unsubstituted or substituted (C5-C8)cycloalkenyl-(C1-C8)alkyl, (C6-C10)bicycloalkyl, unsubstituted or substituted heterocycloalkyl, unsubstituted or substituted heterocycloalkyl-(C1-C8)alkyl, unsubstituted or substituted aryl, unsubstituted or substituted aryl- (C1-C8)alkyl, unsubstituted or substituted heteroaryl, unsubstituted or substituted heteroaryl-(C1-C8)alkyl, cyano, —CORa, —CO2Ra, —CONRaRb, —CONRaNRaRb, —SRa, —SORa, —SO2Ra, —SO2NRaRb, nitro, —NRaRb, —NRaC(O)Rb, —NRaC(O)NRaRb, —NRaC(O)ORa, —NRaSO2Rb, —NRaSO2NRaRb, —NRaNRaRb, —NRaNRaC(O)Rb, —NRaNRaC(O)NRaRb, —NRaNRaC(O)ORa, —ORa, —OC(O)Ra, —OC(O)NRaRb; wherein any (C1-C8)alkyl, (C2-C8)alkenyl, (C2-C8)alkynyl, cycloalkyl, cycloalkenyl, bicycloalkyl, heterocycloalkyl, aryl, or heteroaryl group is optionally substituted by 1, 2 or 3 groups independently selected from the group consisting of —O(C1-C6)alkyl(Rc)1-2, —S(C1-C6)alkyl(Rc)1-2, —(C1-C6)alkyl(Rc)1-2, (C1-C8)alkyl-heterocycloalkyl, (C3-C8)cycloalkyl- heterocycloalkyl, halo, (C1-C6)alkyl, (C3-C8)cycloalkyl, (C5-C8)cycloalkenyl, (C1-C6)haloalkyl, cyano, —CORa, —C02Ra, —CONRaRb, —SRa, —SORa, —SO2Ra, —SO2NRaRb, nitro, —NRaRb, —NRaC(O)Rb, —NRaC(O)NRaRb, —NRaC(O)ORa, —NRaSO2Rb, —NRaSO2NRaRb, —ORa, —OC(O)Ra, —OC(O)NRaRb, heterocycloalkyl, aryl, heteroaryl, aryl(C1-C4)alkyl, and heteroaryl(C1-C4)alkyl;

wherein any aryl or heteroaryl moiety of said aryl, heteroaryl, aryl(C1-C4)alkyl, or heteroaryl(C1-C4)alkyl is optionally substituted by 1, 2 or 3 groups independently selected from the group consisting of halo, (C1-C6)alkyl, (C3-C8)cycloalkyl, (C5-C8)cycloalkenyl, (C1-C6)haloalkyl, cyano, —CORa, —CO2Ra, —CONRaRb, —SRa, —SORa, —SO2Ra, —SO2NRaRb, nitro, —NRaRb, —NRaC(O)Rb, —NRaC(O)NRaRb, —NRaC(O)ORa, —NRaSO2Rb, —NRaSO2NRaRb, —ORa, —OC(O)Ra, and —OC(O)NRaRb; Ra and Rb are each independently hydrogen, (C1-C8)alkyl, (C2-C8)alkenyl, (C2-C8)alkynyl, (C3-C8)cycloalkyl, (C5-C8)cycloalkenyl, (C6-C10)bicycloalkyl, heterocycloalkyl, aryl, heteroaryl, wherein said (d-d)alkyl, (C2-C8)alkenyl, (C2-C8)alkynyl, cycloalkyl, cycloalkenyl, bicycloalkyl, heterocycloalkyl, aryl or heteroaryl group is optionally substituted by 1, 2 or 3 groups

independently selected from halo, hydroxyl, (C1-C4)alkoxy, amino, (C1-C4)alkylamino, ((C1-C4)alkyl)((C1-C4)alkyl)amino, —CO2H, —CO2(C1-C4)alkyl, —CONH2, —CONH(C1-C4)alkyl, —CON((C1-C4)alkyl)((C1-C4)alkyl), —SO2(C1-C4)alkyl, —SO2NH2, —S02NH(C1-C4)alkyl, or —SO2N((C1-C4)alkyl)((C1-C4)alkyl);

or Ra and Rb taken together with the nitrogen to which they are attached represent a 5-8 membered saturated or unsaturated ring, optionally containing an additional heteroatom selected from oxygen, nitrogen, and sulfur, wherein said ring is optionally substituted by 1, 2 or 3 groups independently selected from (C1-C4)alkyl, (C1-C4)haloalkyl, amino, (C1-C4)alkylamino, ((C1-C4)alkyl)((C1-C4)alkyl)amino, hydroxyl, oxo, (C1-C4)alkoxy, and (C1-C4)alkoxy(C1-C4)alkyl, wherein said ring is optionally fused to a (C3-C8)cycloalkyl, heterocycloalkyl, aryl, or heteroaryl ring; or Ra and Rb taken together with the nitrogen to which they are attached represent a 6- to 10-membered bridged bicyclic ring system optionally fused to a (C3-C8)cycloalkyl, heterocycloalkyl, aryl, or heteroaryl ring;

each Rc is independently (C1-C4)alkylamino, —NRaSO2Rb, —SORa, —SO2Ra, —NRaC(O)ORa, —NRaRb, or —CO2Ra; or a solvate or a pharmaceutically acceptable salt thereof.

In particular the inhibitor of EZH2 may be any of the compounds of the formula (I) described in WO2011/140325 or a pharmaceutically acceptable salt thereof. For example the inhibitor of EZH2 may be the compound of formula (I) specified in any one of claims 1 to 9 in WO2011/140325. In particular, the inhibitor of EZH2 may be any one of the compounds of Examples 1 to 131 described in WO2011/140325 or solvates or pharmaceutically acceptable salts thereof. In one preferred embodiment of the invention the inhibitor of EZH2 is the compound of Example 24 of WO2011/140325 or pharmaceutically acceptable salt thereof.

Thus, the inhibitor of EZH2 may be a compound of formula B

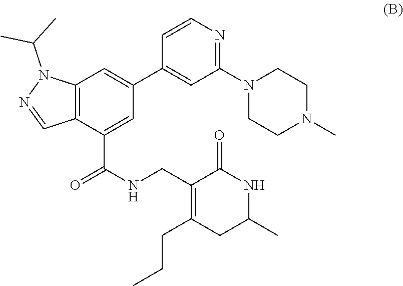 [SMILES: CCCC1=C(CNC(=O)c2cc(-c3ccnc(N4CCN(C)CC4)c3)cc3c2cnn3C(C)C)C(=O)NC(C)C1]

or a solvate or a pharmaceutically acceptable salt thereof. The compound of formula B is also known as GSK343.

In one embodiment of the invention the inhibitor of EZH2 may be any of the inhibitors of EZH2 described in Knutson et al., 2014. In particular, the inhibitor of EZH2 may be the compound EPZ6438 described therein. Thus, the inhibitor of EZH2 is the compound of formula C

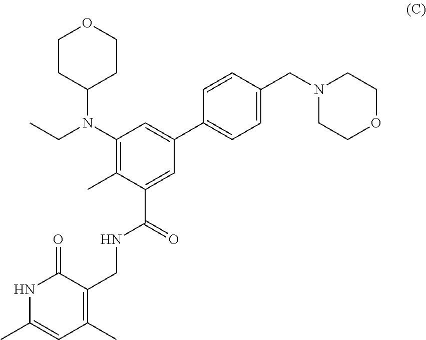 [SMILES: CCN(c1cc(-c2ccc(CN3CCOCC3)cc2)cc(C(=O)NCc2c(C)cc(C)[nH]c2=O)c1C)C1CCOCC1]

or a solvate or a pharmaceutically acceptable salt thereof. The compound of formula C is also known as EPZ6438.

In one embodiment of the invention the inhibitor of EZH2 may be any of the inhibitors of EZH2 described in international patent application WO 2014/062733. For example the inhibitor of EZH2 may be any of the compounds of formulas (I), (Ia), (Ib), (Ic), (Id), (Ie), (Ig), (IA), (I′), (I″), (I″a), (I″b), (I″c), (I″d), (II), (IIa), (IIA), (IIB), (II′), (III), (IIIa), (IIIb), (IIIe), (III′), (IV), (IVa), (IVb), (V), (VI), (VII), (VIIa) and (VIlb) of WO 2014/062733 described therein or solvates or pharmaceutically acceptable salts thereof. In particular, the inhibitor of EZH2 may be any of the compounds of formulas (I), (II), (III), (IVa), (IVb), VI) or (VII) specified in any one of claims 1 to 47 of WO 2014/062733. In particular, the inhibitor of EZH2 may be any one of the compounds 1 to 28 or 101 to 163 described in WO 2014/062733 or solvates or pharmaceutically acceptable salts thereof.

In one embodiment of the invention the inhibitor of EZH2 may be any of the inhibitors of EZH2 described in Knutson et al., 2012. In particular, the inhibitor of EZH2 may be any one of the compounds EPZ004777 or EPZ005687 described therein or solvates or pharmaceutically acceptable salts thereof.

In one embodiment of the invention the inhibitor of EZH2 may be any of the inhibitors of EZH2 described in Garapaty-Rao et al., 2013. In particular, the inhibitor of EZH2 may be any one of the compounds 1, 2 or 3 outlined in Table 1 of Garapaty-Rao et al., 2013 or solvates or pharmaceutically acceptable salts thereof.

In one embodiment of the invention the inhibitor of EZH2 may be any of the inhibitors of EZH2 described in Qi et al., 2012. In particular, the inhibitor of EZH2 may be the compound EI1 described in Qi et al., 2012 or solvates or pharmaceutically acceptable salts thereof.

In one embodiment of the invention the inhibitor of EZH2 may be any of the inhibitors of EZH2 described in McCabe et al., 2012. In particular, the inhibitor of EZH2 may be the compound GSK126 described in McCabe et al., 2012 or solvates or pharmaceutically acceptable salts thereof.

In one embodiment of the invention the inhibitor of EZH2 may be any of the inhibitors of EZH2 described in international patent application WO2011/140324. For example the inhibitor of EZH2 may be a compound of the formula (I) of WO2011/140324 or a pharmaceutically acceptable salt thereof. For example the inhibitor of EZH2 may be the compound of formula (I) specified in any one of claims 1 to 10 of WO2014/172044. In particular, the inhibitor of EZH2 may be any one of the compounds of examples 3 to 373 described in WO2011/140324 or solvates or pharmaceutically acceptable salts thereof.

In one embodiment of the invention the inhibitor of EZH2 may be any of the inhibitors of EZH2 described in international patent application WO2012/005805. For example the inhibitor of EZH2 may be a compound of the formula (I) of WO2012/005805 or a pharmaceutically acceptable salt thereof. For example the inhibitor of EZH2 may be the compound of formula (I) specified in any one of claims 1 to 5 of WO2012/005805. In particular, the inhibitor of EZH2 may be any one of the compounds of examples 1 to 125 described in WO2012/005805 or solvates or pharmaceutically acceptable salts thereof.

In one embodiment of the invention the inhibitor of EZH2 may be any of the inhibitors of EZH2 described in international patent application WO2014/172044. For example the inhibitor of EZH2 may be a compound of the formula (I) of WO2014/172044 or a pharmaceutically acceptable salt thereof. For example the inhibitor of EZH2 may be the compound of formula (I) specified in any one of claims 1 to 46 of WO2014/172044. In particular, the inhibitor of EZH2 may be any one of the compounds 1 to 93 described in WO2014/172044 or solvates or pharmaceutically acceptable salts thereof.

In one embodiment of the invention the inhibitor of EZH2 may be any of the inhibitors of EZH2 described in international patent application WO 2014/144747. For example the inhibitor of EZH2 may be any of the compounds of formulas (I), (Ia), (Ib), (Ic) and (II) described in WO 2014/144747 or solvates or pharmaceutically acceptable salts thereof. For example the inhibitor of EZH2 may be the compound of formula (I), (II), (Ia) specified in any one of claims 1 to 15 of WO 2014/144747. In particular, the inhibitor of EZH2 may be any one of the compounds mentioned in tables 1 and 2 of WO 2014/144747 or solvates or pharmaceutically acceptable salts thereof

In one embodiment of the invention the inhibitor of EZH2 may be any of the inhibitors of EZH2 described in international patent application WO 2014/100646. For example the inhibitor of EZH2 may be any of the compounds of formulas (I), (Ia), (Ib), (Ic), (Id), (II), (IIa), (IIb), (III), (IV) and (Iva) described in WO 2014/100646 or solvates or pharmaceutically acceptable salts thereof. In particular, the inhibitor of EZH2 may be any of the compounds of formulas (I), (Ia), (Ib), (Ic), (Id), (II), (IIa), (IIb), (III), (IV) and (Iva) specified in any one of claims 1 to 20 of WO 2014/100646. In particular, the inhibitor of EZH2 may be any one of the compounds 1 to 238 described in WO 2014/100646 or solvates or pharmaceutically acceptable salts thereof.

In one embodiment of the invention the inhibitor of EZH2 may be any of the inhibitors of EZH2 described in international patent application WO 2014/100665. For example the inhibitor of EZH2 may be any of the compounds of formulas (II), (IIa), (IIb), (IIc), (IId), (III), (IIIa), (IIIb), (IIIc), (IIId), (IV), (Iva), (IVb), (V), (VI), (VIa), (VIb) and (VIc) described in WO 2014/100665 or solvates or pharmaceutically acceptable salts thereof. In particular, the inhibitor of EZH2 may be any of the compounds of formulas (IV), (V), (VI) and (VIc) specified in any one of claims 1 to 8 of WO 2014/100665. In particular, the inhibitor of EZH2 may be any one of the compounds 1 to 23 described in WO 2014/100665, such as compound 1, compound 2 or compound 4 of WO 2014/100665 or solvates or pharmaceutically acceptable salts thereof. The inhibitor of EZH2 may also be anyone of the compounds described in Table 2, Table 3 or Table 4 of WO 2014/100665 or solvates or pharmaceutically acceptable salts thereof.

In one embodiment of the invention the inhibitor of EZH2 may be any of the inhibitors of EZH2 described in international patent application WO 2014/097041. For example the inhibitor of EZH2 may be any of the compounds of formulas (I), (I-A), (I-B), (I-C), (II), (II-A), (II-B), (II-C), (III), (III-A), (III-B), (III-C), (IV), (IV-A), (IV-B) and (IV-C) described in WO 2014/097041 or solvates or pharmaceutically acceptable salts thereof. In particular, the inhibitor of EZH2 may be any of the compounds of formulas (II) and (II-A) specified in any one of claims 1 to 11 of WO 2014/097041. In particular, the inhibitor of EZH2 may be any one of the Examples 2 to 302 described in WO 2014/097041, such as any of examples 1, 53, 58, 253, 229, 66, 76, 77, 90, 143, 107, 108, 112, 113, 114, 116, 123, 124, 126, 128, 131, 132, 133, 134, 217, 145 and 293 of WO 2014/097041, or solvates or pharmaceutically acceptable salts thereof.

In one embodiment of the invention the inhibitor of EZH2 may be any of the inhibitors of EZH2 described in international patent application WO 2014/107277. For example the inhibitor of EZH2 may be any of the compounds of formulas (I) described in WO 2014/107277 or solvates or pharmaceutically acceptable salts thereof. In particular, the inhibitor of EZH2 may be any of the compounds of formula (I) specified in any one of claims 1 to 22 of WO 2014/107277. In particular, the inhibitor of EZH2 may be the compound of any one of the Examples 2 to 20 of WO 2014/107277 or solvates or pharmaceutically acceptable salts thereof.

In one embodiment of the invention the inhibitor of EZH2 may be any of the inhibitors of EZH2 described in international patent application WO 2014/062720. For example the inhibitor of EZH2 may be any of the compounds of formulas (I), (II), (III), (Iva), (IVb), (V), (VI), (Via) and (VII) described in WO 2014/062720. In particular, the inhibitor of EZH2 may be any of compound A, compound B, compound C, compound D, compound E, compound F, compound G or compound H described in WO 2014/062720 or solvates or pharmaceutically acceptable salts thereof.

In one embodiment of the invention the inhibitor of EZH2 may be any of the inhibitors of EZH2 described in international patent application WO 2014/1049488. For example the inhibitor of EZH2 may be any of the compounds of formulas (I), (II), (III), (IV), (V), (VI) and (VII) described in WO 2014/1049488 or solvates or pharmaceutically acceptable salts thereof. In particular, the inhibitor of EZH2 may be any of the compounds of formula (III) specified in any one of claims 1 to 10 of WO 2014/1049488. In particular, the inhibitor of EZH2 may be the compound of any one of the Examples 1 to 150 of WO 2014/1049488 or solvates or pharmaceutically acceptable salts thereof.

In one embodiment of the invention the inhibitor of EZH2 may be any of the inhibitors of EZH2 described in international patent application WO 2013/173441. For example the inhibitor of EZH2 may be any of the compounds of formula (I) described in WO 2013/173441 or solvates or pharmaceutically acceptable salts thereof. In particular, the inhibitor of EZH2 may be any of the compounds of formula (I) specified in any one of claims 1 to 8 of WO 2013/173441. In particular, the inhibitor of EZH2 may be the compound of any one of the Examples 1 to 47 of WO 2013/173441 or solvates or pharmaceutically acceptable salts thereof.

In one embodiment of the invention the inhibitor of EZH2 may be any of the inhibitors of EZH2 described in international patent application WO 2013/039988. For example the inhibitor of EZH2 may be any of the compounds of formulas (I) or (VII) described in WO 2013/039988 or solvates or pharmaceutically acceptable salts thereof. In particular, the inhibitor of EZH2 may be any of the compounds of formula (I) specified in any one of claims 1 to 9 of WO 2013/039988. In particular, the inhibitor of EZH2 may be the compound of any one of the Examples 1 to 144 of WO 2013/039988 or solvates or pharmaceutically acceptable salts thereof.

In one embodiment of the invention the inhibitor of EZH2 may be any of the inhibitors of EZH2 described in international patent application WO 2012/142513. For example the inhibitor of EZH2 may be any of the compounds of formulas (I), (Ia), (Ib), (Ic), (Id), (Ie), (If), (II), (IIa) and (III) described in WO 2012/142513 or solvates or pharmaceutically acceptable salts thereof. In particular, the inhibitor of EZH2 may be any of the compounds of formulas (I), (Ia), (Ie) or (II) specified in any one of claims 1 to 65 of WO 2012/142513. In particular, the inhibitor of EZH2 may be any one of compounds 1 to 418 of WO 2012/142513 or solvates or pharmaceutically acceptable salts thereof.

In one embodiment of the invention the inhibitor of EZH2 may be any of the inhibitors of EZH2 described in international patent application WO 2012/118812. For example the inhibitor of EZH2 may be any of the compounds of formulas (I), (Ia) or (Ib) described in WO 2012/118812 or solvates or pharmaceutically acceptable salts thereof. In particular, the inhibitor of EZH2 may be any of the compounds of formulas (I), (Ia) or (Ib) specified in any one of claims 1 to 33 of WO 2012/118812. In particular, the inhibitor of EZH2 may be any one of compounds A-1 to A-126 described in Table 1 of WO 2012/118812, compounds B-1 to B-164 described in Table 2 of WO 2012/118812, compounds C-1 to C-35 described in Table 3 of WO 2012/118812, compounds E-1 to E2 described in Table 5 of WO 2012/118812, compounds F-1 to F-2 described in Table 6 of WO 2012/118812 or solvates or pharmaceutically acceptable salts thereof.

In one embodiment of the invention the inhibitor of EZH2 may be any of the inhibitors of EZH2 described in international patent application WO 2012/082436. For example the inhibitor of EZH2 may be any of the compounds of formula (I) described in WO 2012/082436 or solvates or pharmaceutically acceptable salts thereof. In particular, the inhibitor of EZH2 may be any of the compounds of formula (I) specified in any one of claims 1 to 94 of WO 2012/082436. In particular, the inhibitor of EZH2 may be any one of the compounds for which the structure is provided on p. 24-30 or p. 59-71 in WO 2012/082436 or solvates or pharmaceutically acceptable salts thereof. The inhibitor os EZH2 may also be any one of the compounds 5, 9, 38, 64, 81, 86, 92, 94, 96, 98, 114, 116, 118, 125, 129, 131, 143, 145, 149, 152, 154, 159, 163, 167, 169, 173, 179, 183, 185, 190, 195, 199, 201, 206, 209, 213, 223 or 300-382 described in WO 2012/082436 or solvates or pharmaceutically acceptable salts thereof.

In one embodiment of the invention the inhibitor of EZH2 may be any of the inhibitors of EZH2 described in international patent application WO 2012/075080. For example the inhibitor of EZH2 may be any of the compounds of formula (I) described in WO 2012/075080 or solvates or pharmaceutically acceptable salts thereof. In particular, the inhibitor of EZH2 may be any of the compounds of formula (I) specified in any one of claims 1 to 6 of WO 2012/075080. In particular, the inhibitor of EZH2 may be any one of the compounds of Examples 1 to 25 of WO 2012/075080 or solvates or pharmaceutically acceptable salts thereof.

In one embodiment of the invention the inhibitor of EZH2 may be any of the inhibitors of EZH2 described in international patent application WO2012/034132. For example the inhibitor of EZH2 may be the compound 75 described in WO2012/034132 or solvates or pharmaceutically acceptable salts thereof.

Cancer

The present invention relates to an inhibitor of EZH2 for use in the treatment of cancer. Said inhibitor of EZH2 may be any one of the inhibitors of EZH2 described herein above in the section “Inhibitor of EZH2”. Said cancer is preferably characterized by expression of mutated histone H3 having a mutation of amino acid number 27 and/or by a mutation in at least one gene encoding histone H3, wherein the mutated histone H3 gene encodes mutated histone H3 having a mutation of amino acid number 27. Said mutation may be a mutation from lysine to any other amino acid, for example a mutation from lysine to any other amino acid, wherein the side chain of the amino acid does not comprise an amine group. For example, the mutation may be a mutation from lysine to any amino acid having a non-polar or hydrophobic side chain. Preferably said mutation of amino acid number 27 is a mutation from lysine to methionine.

In particular said cancer may be characterised by expression of mutated histone H3 having a mutation of amino acid number 27 from lysine to any other amino acid. Preferably, said cancer is characterised by expression of mutated histone H3 having a mutation of amino acid number 27 from lysine to either isoleucine or methionine. In one preferred embodiment of the invention the cancer is characterised by expression of mutated histone H3 having a mutation of amino acid number 27 from lysine to methionine.

The cancer may also be characterised by a mutation in at least one gene encoding histone H3, wherein the mutated histone H3 gene encodes mutated histone H3 having a mutation of amino acid number 27 from lysine to any other amino acid. Preferably, said cancer is characterised by a mutation in at least one gene encoding histone H3, wherein the mutated histone H3 gene encodes mutated histone H3 having a mutation of amino acid number 27 from lysine to either isoleucine or methionine. In one preferred embodiment of the invention the cancer is characterised by a mutation in at least one gene encoding histone H3, wherein the mutated histone H3 gene encodes mutated histone H3 having a mutation of amino acid number 27 from lysine to methionine

The tail of histone H3 contains several lysine residues, which may be methylated. Methylation of histone H3 is involved in epigenetic downregulation of gene expression. EZH2 is the enzymatic component of the Polycomb repressive complex 2 (PRC2), which represses gene expression by methylating lysine 27 of histone H3.

In mutated histone H3 having a mutation of amino acid number 27 methylation of residue 27 is not possible. It has been shown that expression of mutant histone H3 having a mutation of amino acid number 27 generally reduces the K27 methylation of histone H3.

The term “H3K27” as used herein refers to the amino acid number 27 (lysine) of histone H3. Thus, the cancer may be a cancer expressing mutated histone H3 mutated in H3K27.

The term “H3K27X” as used herein refers to the histone H3, wherein the amino acid at position 27 is mutated from lysine to another amino acid. Thus, the cancer according to the invention may be characterized by expression of H3K27X and/or the cancer is characterised by a mutation in at least one gene encoding histone H3, wherein the mutated histone H3 gene encodes H3K27X.

The term “H3K27M” as used herein refers to the histone H3, wherein the amino acid at position 27 is mutated from lysine to methionine. Thus, the cancer according to the invention may be characterized by expression of H3K27M and/or the cancer is characterised by a mutation in at least one gene encoding histone H3, wherein the mutated histone H3 gene encodes H3K27M.

The term “H3K27I” as used herein refers to the histone H3, wherein the amino acid at position 27 is mutated from lysine to isoleucine. Thus, the cancer according to the invention may be characterized by expression of H3K27I and/or the cancer is characterised by a mutation in at least one gene encoding histone H3, wherein the mutated histone H3 gene encodes H3K27I.

In human beings several different histone H3s are expressed, including histone H3.1, histone H3.2 and histone H3.3. Thus, the cancer may be characterized by expression of mutated histone H3.1, having a mutation of amino acid number 27, for example having a mutation of amino acid number 27 from lysine to methionine. Thus, the cancer may be characterized by expression of a protein of SEQ ID NO:2, wherein amino acid 27 is not lysine. In particular, the cancer may be characterized by expression of a protein of SEQ ID NO:2, wherein amino acid 27 is methionine or isoleucine, and in particular amino acid 27 may be methionine. The cancer may also be characterised by a mutation in at least one gene encoding histone H3.1, wherein the mutated histone H3.1 gene encodes a protein of SEQ ID NO:2, wherein amino acid 27 is not lysine, for example amino acid 27 may be methionine or isoleucine, in particular amino acid 27 may be methionine.

The cancer may also be characterized by expression of mutated histone H3.2, having a mutation of amino acid number 27, for example having a mutation of amino acid number 27 from lysing to methionine or isoleucine. The cancer may also be characterised by a mutation in at least one gene encoding histone H3.2, wherein the mutated histone H3.2 gene encodes histone H3.2, wherein amino acid 27 is not lysine, for example amino acid 27 may be methionine or isoleucine, in particular amino acid 27 may be methionine.

The cancer may be characterized by expression of mutated histone H3.3, having a mutation of amino acid number 27, for example having a mutation of amino acid number 27 from lysing to methionine. Thus, the cancer may be characterized by expression of a protein of SEQ ID NO:1, wherein amino acid 27 is not lysine. In particular, the cancer may be characterized by expression of a protein of SEQ ID NO:1, wherein amino acid 27 is methionine or isoleucine, and in particular amino acid 27 may be methionine. The cancer may also be characterised by a mutation in at least one gene encoding histone H3.3, wherein the mutated histone H3.3 gene encodes a protein of SEQ ID NO:1, wherein amino acid 27 is not lysine, for example amino acid 27 may be methionine or isoleucine, in particular amino acid 27 may be methionine.

In particular the cancer may also be characterised expression of a protein of SEQ ID NO:3 and/or the cancer may be characterised by a mutation in at least one gene encoding histone H3.3, wherein the mutated histone H3.3 gene encodes a protein of SEQ ID NO:3. In general, the cancer may express both wild type and mutant histone H3. Thus, the cancer may express wild type histone H3.1 and H3.2, and mutated histone H3.3 having a mutation of amino acid number 27. The cancer may also express wild type histone H3.2 and H3.3, and mutated histone H3.1 having a mutation of amino acid number 27. The cancer may express wild type histone H3.1 and H3.3, and mutated histone H3.2 having a mutation of amino acid number 27.

Since each variant of histone H3 is encoded by several genes in human beings, the cancer may also express both wild type and mutant histone H3.1. The cancer may also express both wild type and mutant histone H3.2. The cancer may also express both wild type and mutant H3.3.

In general said cancer carries a mutation in at least one of the genes encoding histone H3. Histone H3s are coded by several genes in the human genome, including:

H3.1 is encoded by the following genes: HIST1H3A, HIST1H3B, HIST1H3C, HIST1H3D, HIST1H3E, HIST1H3F, HIST1H3G, HIST1H3H, HIST1H31, HIST1H3J.

Thus, the cancer may be a cancer wherein at least one of the genes HIST1H3A, HIST1H3B, HIST1H3C, HIST1H3D, HIST1H3E, HIST1H3F, HIST1H3G, HIST1H3H, HIST1H31, or HIST1H3J carries a mutation so that said gene encodes a mutated histone H3.1 having a mutation of amino acid number 27, for example having a mutation of amino acid number 27 from lysine to methionine or isoleucine, such as having a mutation of amino acid number 27 from lysine to methionine

H3.2 is encoded by the following genes: HIST2H3A, HIST2H3C, HIST2H3D.

Thus, the cancer may be a cancer wherein at least one of the genes HIST2H3A, HIST2H3C, or HIST2H3D carries a mutation so that said gene encodes a mutated histone H3.2 having a mutation of amino acid number 27, for example having a mutation of amino acid number 27 from lysine to methionine or isoleucine, such as having a mutation of amino acid number 27 from lysine to methionine.

H3.3 is encoded by the following genes: H3F3A, H3F3B.

Thus, the cancer may be a cancer wherein at least one of the genes H3F3A or H3F3B carries a mutation so that said gene encodes a mutated histone H3.3 having a mutation of amino acid number 27, for example having a mutation of amino acid number 27 from lysine to methionine or isoleucine, such as having a mutation of amino acid number 27 from lysine to methionine.

The cancer to be treated with the inhibitor of EZH2 may also be a cancer, which is characterized by the presence of a gene encoding p16^{INK4A}. p16^{INK4A} is also known as p16. In particular the cancer may be characterized by the presence of a gene encoding wild type p16^{INK4A}, such as p16^{INK4A} of SEQ ID NO:5. Said cancer may be characterized both by the presence of a gene encoding p16^{INK34A}, e.g. p16^{INK4A} of SEQ ID NO:5 and by expression of a mutated histone H3 having a mutation of amino acid number 27 as outlined above. Alternatively, the cancer may be characterized only by of the presence of a gene encoding p16^{INK4A} or only by expression of a mutated histone H3 having a mutation of amino acid number 27.

Thus, the cancer may be characterised by containing an intact p16 locus. The p16 locus is also known as the INK4A locus or as CDKN2A. It is thus preferred that the p16 locus or the CDKN2A locus in said cancer is not deleted.

In a preferred embodiment of the invention, the cancer further is characterised by essentially no expression of p16^{INK4A}. In particular, the cancer may be characterised by no detectable expression of p16^{INK4A} Detection may preferably be performed by Western Blotting for example as described in Example 1 below.

p16^{INK4A} may in particular be the protein of SEQ ID NO:5. Thus, the cancer may be characterised by no detectable expression of p16^{INK4A} of SEQ ID NO:5.

The cancer may be any type of cancer characterised by expression of a mutated histone H3 having a mutation of amino acid number 27 as outlined above and/or by containing a gene encoding p16^{INK4A}.

Thus, the cancer may for example be selected from the group consisting of: diffuse intrinsic pontine glioma, colon carcinoma, breast cancer, pancreatic cancer, ovarian cancer, prostate cancer, fibrosarcoma, myxosarcoma, liposarcoma, chondrosarcoma, osteogenic sarcoma, chordoma, angiosarcoma, endotheliosarcoma, lymphangeosarcoma, lymphangeoendothelia sarcoma, synovioma, mesothelioma, Ewing's sarcoma, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioblastomas, neuronomas, craniopharingiomas, schwannomas, glioma, astrocytoma, medulloblastoma, craniopharyngioma, ependymoma, pinealoma, hemangioblastoma, acoustic neuroama, oligodendroglioma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, childhood-null acute lymphoid leukemia (ALL), thymic ALL, B-cell ALL, acute myeloid leukemia, myelomonocytoid leukemia, acute megakaryocytoid leukemia, Burkitt's lymphoma, acute myeloid leukemia, chronic myeloid leukemia, and T cell leukemia, small and large non-small cell lung carcinoma, acute granulocytic leukemia, germ cell tumors, endometrial cancer, gastric cancer, cancer of the head and neck, chronic lymphoid leukemia, hairy cell leukemia and thyroid cancer.

In one preferred embodiment of the invention the cancer is diffuse intrinsic pontine glioma.

Method of Predicting Efficacy of Treatment

It is also an aspect of the present invention to provide a method for predicting the efficacy of treatment of a cancer with an inhibitor of EZH2 in an individual in need thereof, said method comprising the steps of
i) providing a sample comprising cells of said cancer from said individual,
ii) determining whether said cells contain a gene encoding p16^{INK4A}, for example determining whether said cells contain a gene encoding p16^{INK4A} of SEQ ID NO:5,
   wherein the presence of a gene encoding p16^{INK4A} (e.g. p16^{INK4A} of SEQ ID NO:5) in said cells is indicative of efficacy of treatment of the cancer in said individual with an inhibitor of EZH2. Said inhibitor of EZH2 may be any of the inhibitors of EZH2 described herein above in the section “Inhibitor of EZH2”. The cancer may be any cancer, however preferably the cancer may be any of the cancers described herein above in the section “Cancer”.

Thus the cancer may in particular be a cancer characterised by expression of mutated histone H3 having a mutation of amino acid number 27 and/or characterised by a mutation in at least one gene encoding histone H3, wherein the mutated histone H3 gene encodes mutated histone H3 having a mutation of amino acid number 27.

The cancer may also be a diffuse intrinsic pontine glioma. In particular, the cancer may be a diffuse intrinsic pontine glioma characterised by expression of mutated histone H3 having a mutation of amino acid number 27 and/or characterised by a mutation in at least one gene encoding histone H3, wherein the mutated histone H3 gene encodes mutated histone H3 having a mutation of amino acid number 27.

It is also an aspect of the invention to provide a method for treatment of cancer in an individual in need thereof, wherein the method comprises the steps of:
iii) Obtaining information of whether cells of the cancer from said individual comprises a gene encoding p16^{INK4A}; and
iv) if said cancer cells contain a gene encoding p16^{INK4A} then administering a therapeutically effective amount of said inhibitor of EZH2 to said individual thereby treating cancer in said individual. Said inhibitor of EZH2 may be any of the inhibitors of EZH2 described herein above in the section “Inhibitor of EZH2”. The cancer may be any cancer, however preferably the cancer may be any of the cancers described herein above in the section “Cancer”. If the cancer does not contain a gene encoding p16^{INK4A} then another treatment than administration of an inhibitor of EZH2 may be preferred.

Said gene encoding p16^{INK4A}, is preferably a gene encoding wild type p16^{INK4A}. In particular said gene encoding p16^{INK4A} is a gene encoding p16^{INK4A} of SEQ ID NO:5. Thus, if the cancer contains an intact p16 locus, e.g. if the p16 locus in said cancer is not deleted, then this may be indicative of efficacy of treatment of the cancer with an inhibitor of EZH2.

Treatment of Cancer

It is also an aspect of the invention to provide a method for treatment of cancer comprising administering a therapeutically effective amount of an inhibitor of EZH2 to an individual in need thereof. Said inhibitor may be any of the inhibitors described herein above in the section “Inhibitor of EZH2”. Said cancer is preferably a cancer characterised by expression of mutated histone H3 having a mutation of amino acid number 27, and may be any of the cancers described herein above in the section “Cancer”.

The term “treatment” as used herein may refer to ameliorating treatment and/or curative treatment and/or treatment reducing the effects of the cancer and/or treatment reducing the growth of the cancer or any other kind of treatment.

The instant compounds can be combined with or co-administered with other therapeutic agents, particularly agents that may enhance the activity or time of disposition of the compounds. Combination therapies according to the invention comprise the administration of at least one compound of the invention and the use of at least one other treatment method. In one embodiment, combination therapies according to the invention comprise the administration of at least one compound of the invention and surgical therapy. In one embodiment, combination therapies according to the invention comprise the administration of at least one compound of the invention and radiotherapy. In one embodiment, combination therapies according to the invention comprise the administration of at least one compound of the invention and at least one supportive care agent (e.g., at least one anti-emetic agent). In one embodiment, combination therapies according to the present invention comprise the administration of at least one compound of the invention (i.e. at least one inhibitor of EZH2) and at least one other chemotherapeutic agent. In one particular embodiment, the invention comprises the administration of at least one compound of the invention and at least one anti-neoplastic agent.

Typically, any anti-neoplastic agent that has activity versus a susceptible tumor being treated may be co-administered in the treatment of specified cancers in the present invention. Examples of such agents can be found in Cancer Principles and Practice of Oncology by V. T. Devita and S. Hellman (editors), 6th edition (Feb. 15, 2001), Lippincott Williams & Wilkins Publishers. A person of ordinary skill in the art would be able to discern which combinations of agents would be useful based on the particular characteristics of the drugs and the cancer involved.

The inhibitor of EZH2 may be administered in the form of a pharmaceutical composition. Pharmaceutical compositions may be presented in unit dose forms containing a predetermined amount of inhibitor of EZH2 per unit dose. Such a unit may contain, for example, 0.5 mg to 1 g, preferably 1 mg to 700 mg, more preferably 5 mg to 100 mg of inhibitor of EZH2, depending on the route of administration and the age, weight and condition of the patient, or pharmaceutical compositions may be presented in unit dose forms containing a predetermined amount of active ingredient per unit dose. Preferred unit dosage compositions are those containing a daily dose or sub-dose, as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association a compound of formal (I) with the carrier(s) or excipient(s).

Pharmaceutical compositions adapted for oral administration may be presented as discrete units such as capsules or tablets; powders or granules; solutions or suspensions in aqueous or nonaqueous liquids; edible foams or whips; or oil-in-water liquid emulsions or water-in-oil liquid emulsions.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of a compound of formula (I). Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit pharmaceutical compositions for oral administration can be microencapsulated. The formulation can also be prepared to prolong or sustain the release as for example by coating or embedding particulate material in polymers, wax or the like.

Pharmaceutical compositions adapted for rectal administration may be presented as suppositories or as enemas.

Pharmaceutical compositions adapted for vaginal administration may be presented as pessaries, tampons, creams, gels, pastes, foams or spray formulations.

Pharmaceutical formulations adapted for parenteral administration include aqueous and nonaqueous sterile injection solutions which may contain anti-oxidants, buffers, bacteriostats and solutes which render the composition isotonic with the blood of the intended recipient; and aqueous and nonaqueous sterile suspensions which may include suspending agents and thickening agents. The pharmaceutical compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the pharmaceutical compositions may include other agents conventional in the art having regard to the type of formulation in question, for example those suitable for oral administration may include flavouring agents.

A therapeutically effective amount of the inhibitor of EZH2 will depend upon a number of factors including, for example, the age and weight of the intended recipient, the precise condition requiring treatment and its severity, the nature of the formulation, and the route of administration, and will ultimately be at the discretion of the attendant prescribing the medication.

However, an effective amount of inhibitor of EZH2 will generally be in the range of 0.001 to 100 mg/kg body weight of recipient per day, for example in the range of 0.01 to 10 mg/kg body weight per day. For a 70 kg adult mammal, the actual amount per day may for example be from 7 to 700 mg and this amount may be given in a single dose per day or in a number (such as two, three, four, five or six) of sub-doses per day such that the total daily dose is the same. An effective amount of a salt or solvate, etc., may be determined as a proportion of the effective amount of the inhibitor of EZH2.

The methods of the invention may comprise obtaining information of whether the cells of the cancer to be treated expresses p16^{INK4A} and administering an inhibitor of EZH2 to the individual if the cells expresses low levels of p16^{INK4A}.

Sequences

TABLE 1

| | |
|---|---|
| SEQ ID NO: 1 | Amino acid sequence of wild type human |
| | histone H3 (canonical H3.3). |
| SEQ ID NO: 2 | Amino acid sequence of wild type human |
| | histone H3 variant H3.1 |
| SEQ ID NO: 3 | Amino acid sequence of human K27M mutant |
| | histone H3 |
| SEQ ID NO: 4 | Amino acid sequence of human EZH2 |
| SEQ ID NO: 5 | Amino acid sequence of human p16^{INK4A} |

SEQ ID NO: 1 - H3.3
>gi|4504279|ref|NP 002098.1| histone H3.3
[Homo sapiens]
MARTKQTARKSTGGKAPRKQLATKAARKSAPSTGGVKKPHRYRPGTVAL
REIRRYQKSTELLIRKLPFQRLVREIAQDFKTDLREQSAAIGALQEASE
AYLVGLFEDTNLCAIHAKRVTIMPKDIQLARRIRGERA
SEQ ID NO: 2 - H3.1
>gi|4504281|ref|NP 003520.1| histone H3.1
[Homo sapiens]
MARTKQTARKSTGGKAPRKQLATKAARKSAPATGGVKKPHRYRPGTVAL
REIRRYQKSTELLIRKLPFQRLVREIAQDFKTDLREQSSAVMALQEACE
AYLVGLFEDTNLCAIHAKRVTIMPKDIQLARRIRGERA
SEQ ID NO: 3 - K27M mutant of H3.3
>gi|4504279|ref|NP 002098.1| histone H3.3
[Homo sapiens]
MARTKQTARKSTGGKAPRKQLATKAARMSAPSTGGVKKPHRYRPGTVAL
REIRRYQKSTELLIRKLPFQRLVREIAQDFKTDLREQSAAIGALQEASE
AYLVGLFEDTNLCAIHAKRVTIMPKDIQLARRIRGERA
SEQ ID NO: 4 - EZH2
>gi|322506097|ref|NP 001190176.1| histone-lysine
N-methyltransferase EZH2 isoform c [Homo sapiens]
MGQTGKKSEKGPVCWRKRVKSEYMRLRQLKRERRADEVKSMESSNRQKI
LERTEILNQEWKQRRIQPVHILTSVSSLRGTRECSVTSDLDEPTQVIPL
KTLNAVASVPIMYSWSPLQQNFMVEDETVLHNIPYMGDEVLDQDGTFIE
ELIKNYDGKVHGDRECGFINDEIFVELVNALGQYNDDDDDDDGDDPEER
EEKQKDLEDHRDDKESRPPRKFPSDKIFEAISSMFPDKGTAEELKEKYK
ELTEQQLPGALPPECTPNIDGPNAKSVQREQSLHSFHTLFCRRCFKYDC
FLHPFHATPNTYKRKNTETALDNKPCGPQCYQHLEGAKEFAAALTAERI
KTPPKRPGGRRRGRLPNNSSRPSTPTINVLESKDTDSDREAGTETGGEN
NDKEEEEKKDETSSSSEANSRCQTPIKMKPNIEPPENVEWSGAEASMFR
VLIGTYYDNFCAIARLIGTKTCRQVYEFRVKESSIIAPAPAEDVDTPPR
KKKRKHRLWAAHCRKIQLKKDGSSNHVYNYQPCDHPRQPCDSSCPCVIA
QNFCEKFCQCSSECQNRFPGCRCKAQCNTKQCPCYLAVRECDPDLCLTC
GAADHWDSKNVSCKNCSIQRGSKKHLLLAPSDVAGWGIFIKDPVQKNEF
ISEYCGEIISQDEADRRGKVYDKYMCSFLENLNNDFVVDATRKGNKIRF
ANHSVNPNCYAKVMMVNGDHRIGIFAKRAIQTGEELFFDYRYSQADALK
YVGIEREMEIP
SEQ ID NO: 5 - p16^{INK4A}
>gi|4502749|ref|NP 000068.1| cyclin-dependent
kinase inhibitor 2A isoform p16INK4a
[Homo sapiens]
MEPAAGSSMEPSADWLATAAARGRVEEVRALLEAGALPNAPNSYGRRPI
QVMMMGSARVAELLLLHGAEPNCADPATLTRPVHDAAREGFLDTLVVLH
RAGARLDVRDAWGRLPVDLAEELGHRDVARYLRAAAGGTRGSNHARIDA
AEGPSDIPD

TABLE 2
ChIP-qPCR primers

| Primer | | |
|---|---|---|
| name | 5′ primer | 3′ primer |
| p16_1 | CACACTCTGCTCCTG | GCAGTGTTTTCAGGG |
| | ACCTG | GTGTT |
| | [SEQ ID NO: 6] | [SEQ ID NO: 7] |
| p16_2 | GCAGCAGCAACAACA | CCATCTGCAGTCGGG |
| | AAAAC | TAACT |
| | [SEQ ID NO: 8] | [SEQ ID NO: 9] |
| Gabra5 | GGAACGTCCCTCTGC | CAGACAGTAGCTCCC |
| | CTAG | ATGCC |
| | [SEQ ID NO: 10] | [SEQ ID NO: 11] |
| Igf2bp3 | CATTGTGAAAAGGGG | TGAAAGCCAAGAGGA |
| | TCGTT | GAGGA |
| | [SEQ ID NO: 12] | [SEQ ID NO: 13] |
| Stk31 | ACAGTCAAGTGCTGC | TCCAACCTCAACACT |
| | AAAAGA | GTCCT |
| | [SEQ ID NO: 14] | [SEQ ID NO: 15] |
| Tacstd2 | ACATCCTCATTGAGT | CAGCTCGGAGTCTAG |
| | TGCGC | GTCAG |
| | [SEQ ID NO: 16] | [SEQ ID NO: 17] |

### EXAMPLES

The invention is further illustrated by the following examples, which however should not be construed as being limiting for the invention.

### Example 1

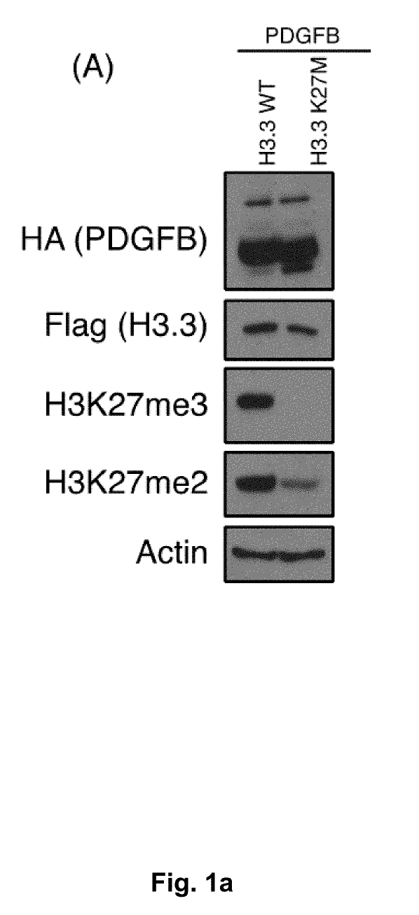

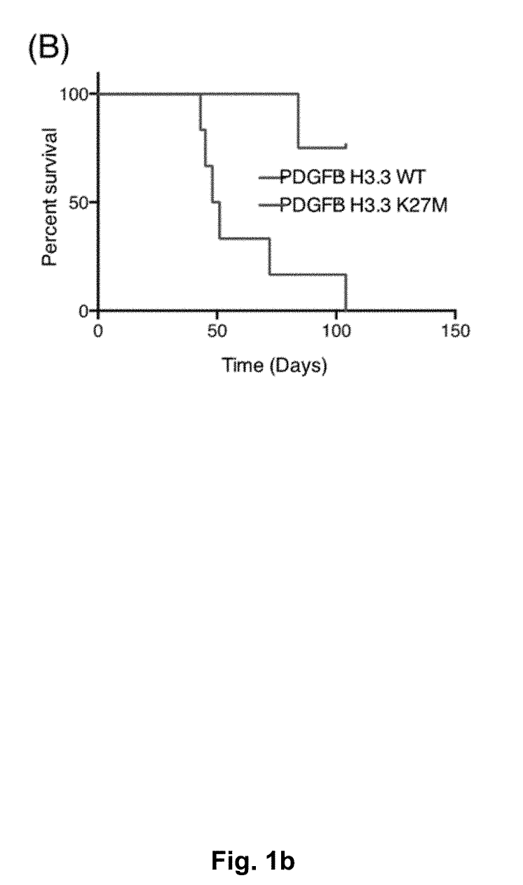

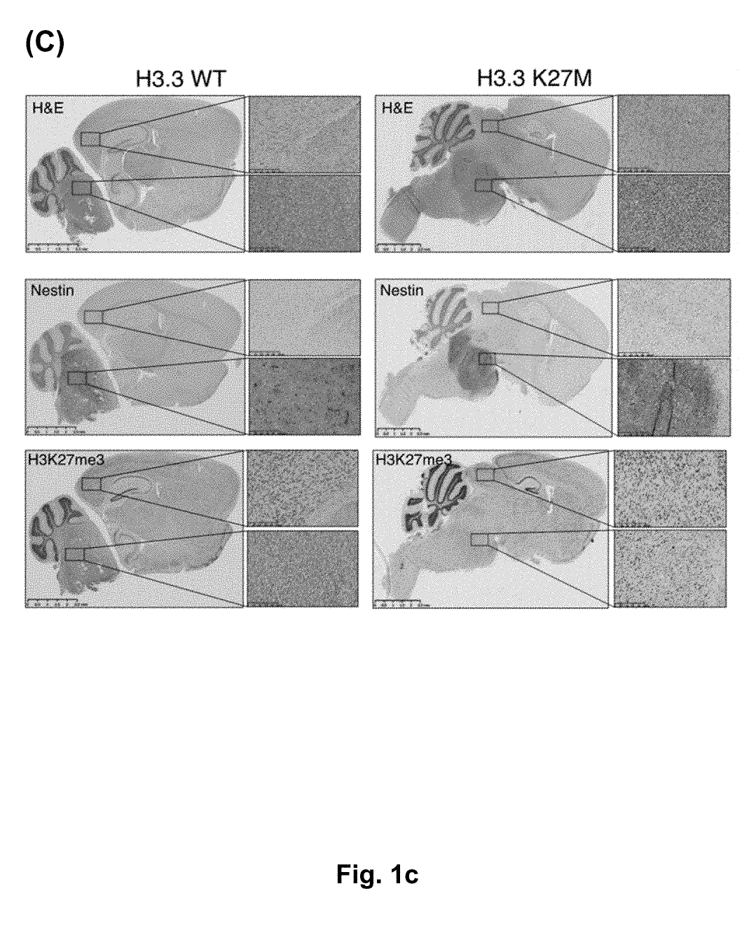

Increased platelet-derived growth factor (PDGF) signaling is frequently associated with H3K27M mutation in DIPGs. Whole-exome sequencing studies have identified recurrent driver mutations in H3F3A and HIST1H3B, leading to the expression of histone H3 in which lysine 27 is substituted with methionine (H3K27M) in nearly 80% of DIPG. To better understand the role of K27M mutation in DIPGs a mouse DIPG model was developed, where we stably co-expressed PDGFB and H3.3K27M in mouse neural stem cells (NSCs). NSCs expressing H3.3K27M showed global reduction of H3K27me3 and H3K27me2 levels (FIG. 1A) and when injected in mouse pons formed tumors much faster than the NSCs expressing wild-type (WT) H3.3 (FIG. 1B). This demonstrates that H3.3K27M can potentiate PDGFB mediated tumor development. Immunohistochemistry of the brain of tumor bearing mice confirmed the localization of tumor in the pons. Tumor formed by PDGFB/H3.3K27M NSCs showed presence of undifferentiated cells (Nestin positive cells) and a considerable reduction in H3K27me3 levels (FIG. 1C).

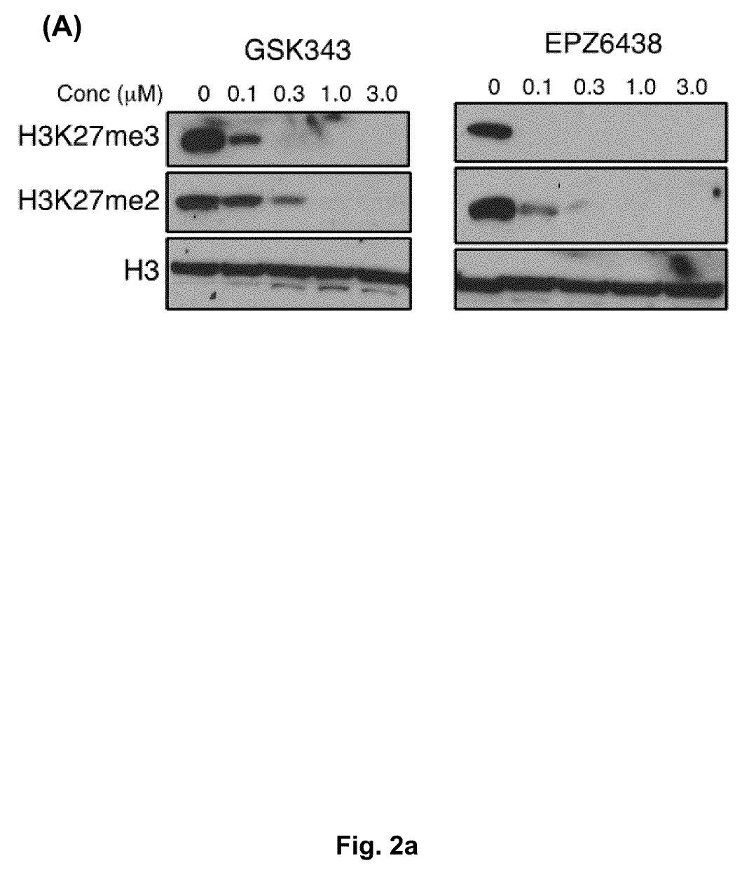

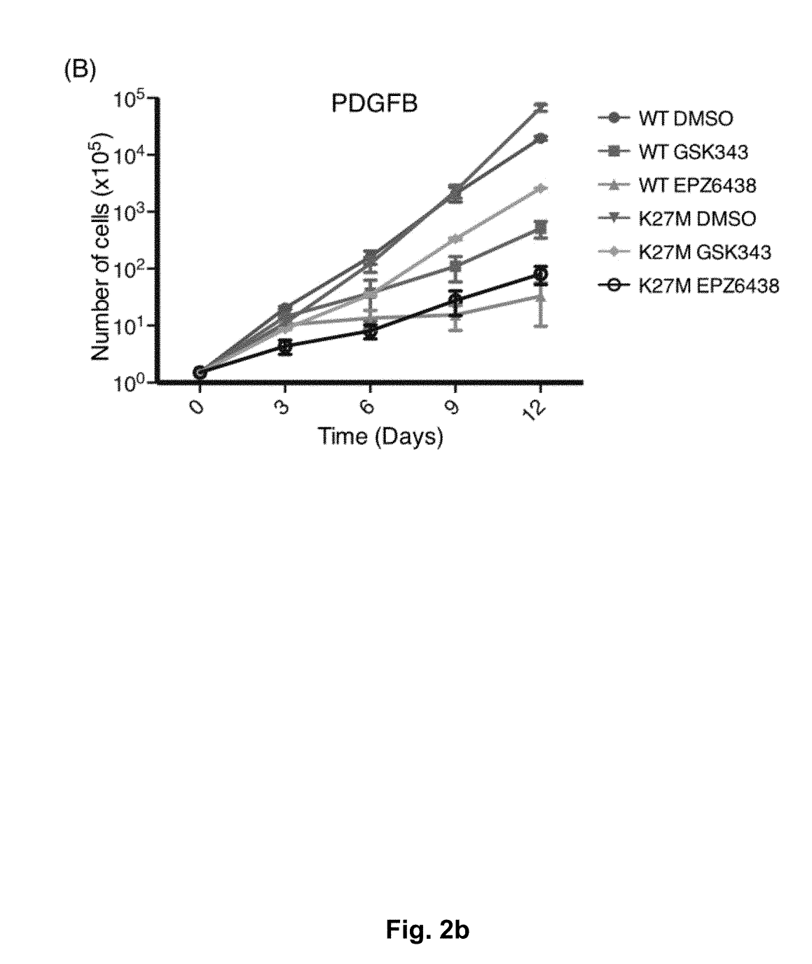

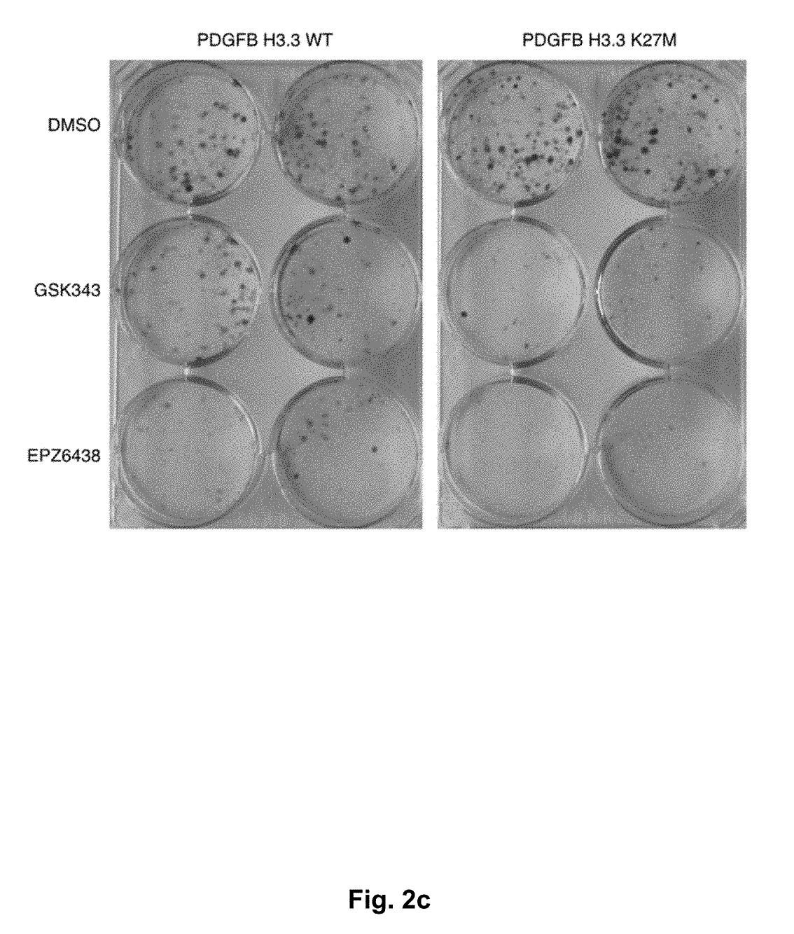

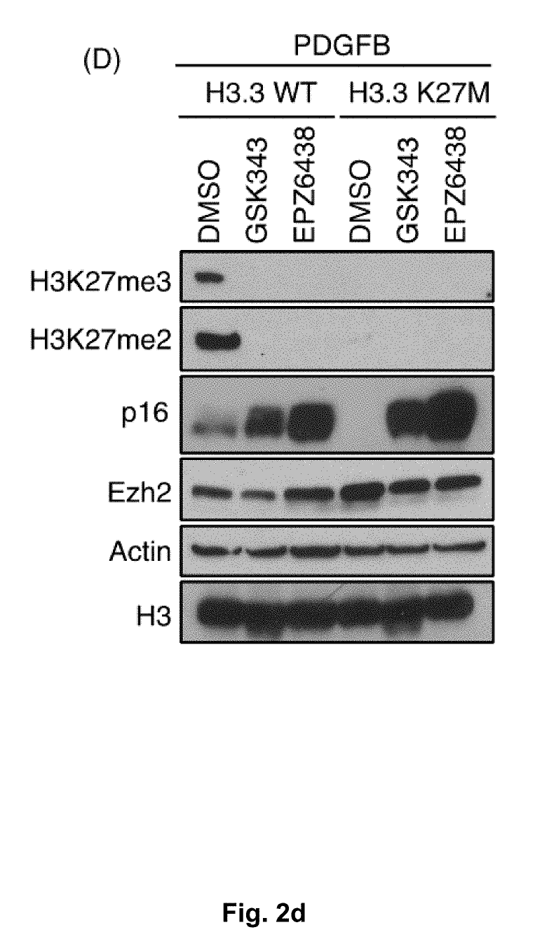

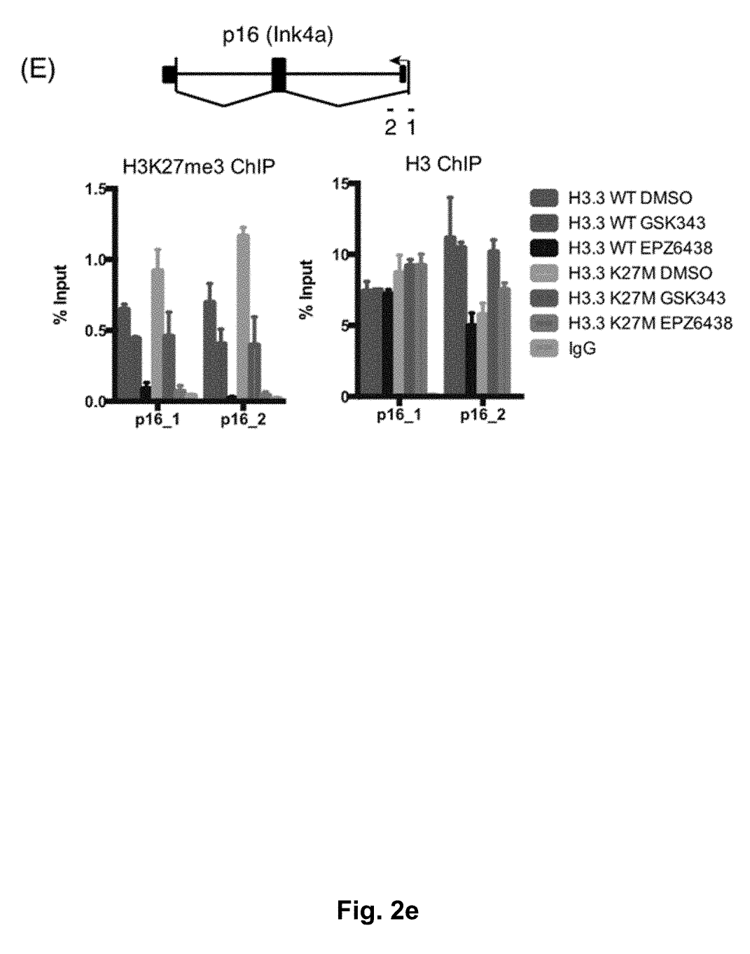

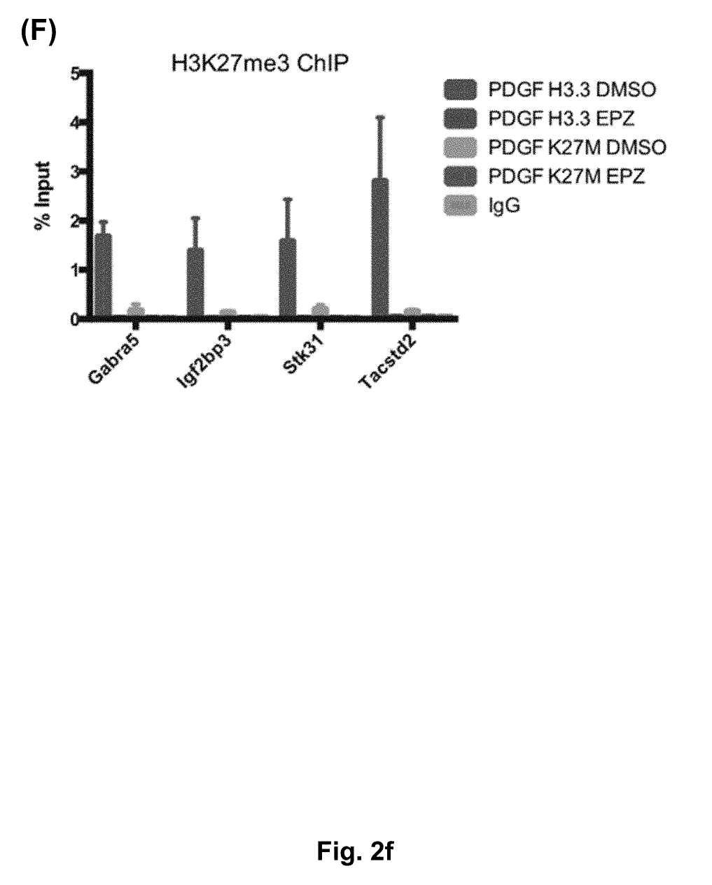

The mouse DIPG cells transformed by H3K27M and PDGFB were treated with two different inhibitors of EZH2 (GSK343 and EPZ6438). Both GSK343 and EPZ6438 are potent and highly selective EZH2 inhibitors with EPZ6438 being more potent (FIG. 2A). Upon Ezh2 inhibitor treatment, PDGFB NSCs expressing either WT or K27M mutant H3.3 showed reduced proliferation as well as formed fewer colonies in a colony formation assay (FIGS. 2B and 2C) surprisingly demonstrating that the residual H3K27me3 in PDGFB/H3.3K27M NSCs is required for DIPG tumor cells growth.

p16^{INK4A} is a tumor suppressor protein that acts as a cell cycle inhibitor and is a target for PRC2-mediated repression in normal cells as well as in tumors. Interestingly, PDGFB/H3.3K27M NSCs showed increased H3K27me3 enrichment at the Ink4a locus associated with reduced p16^{Ink4A} levels (FIGS. 2D and 2E), and treatment of the cells with the two different EZH2 inhibitors resulted in reduced H3K27me3 levels and corresponding increase in p16^{Ink4a} levels (FIGS. 2D and 2E). The enrichment of H3K27me3 levels at the Ink4a locus is somewhat surprising, because of the global reduction of H3K27me3 levels in the transformed NSC cells. In fact H3K27me3 levels are in general reduced on PRC2 target genes in PDGFB/H3.3K27M NSCs, as for instance on Gabra5, Igf2bp3, Stk31, and Tacstd2 (FIG. 2F).

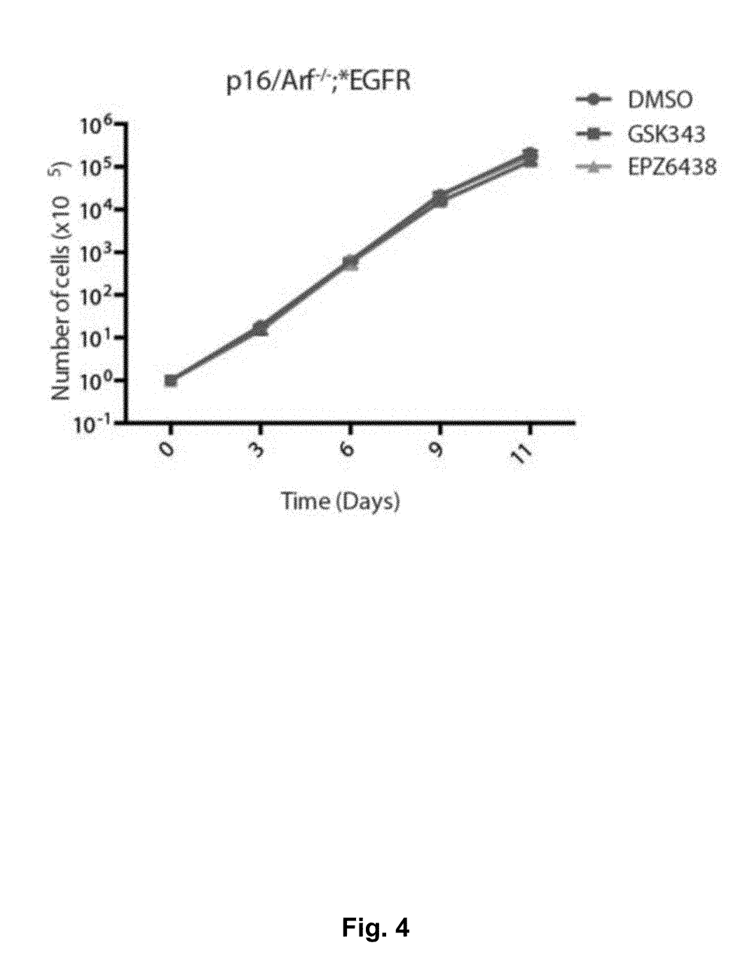

A mouse model representing adult classical subtype of GBM subtypes was investigated. Ink4a−/−/Arf−/− NSCs expressing constitutive active epidermal growth factor receptor (*EGFR) were treated with EZH2 inhibitors (GSK343 and EPZ6438), however the treatment did not affect their proliferation (see FIG. 4).

Taken together, the present inventions shows that although tumor cells expressing H3K27M are characterized by a global reduction of H3K27me3 levels, which is widely believed to be mechanistically important for tumorigenesis, they are still sensitive to EZH2 inhibition. We also demonstrated that the effect of Ezh2 inhibition on mouse DIPG cells is mediated by genes such as Ink4a that paradoxically shows increased H3K27me3 enrichment in PDGFB/H3.3K27M NSCs and upon Ezh2 inhibitor treatment shows increased expression associated with loss of associated H3K27me3. These results demonstrates that inhibitors of EZH2 would be useful for the of DIPG patients in which the tumors express H3K27M.

Materials and Methods

Expression Plasmids

The PDGFB expression vector (pCDNA-PDGFB) was a kind gift from Lene Uhrbom (Jiang et al., 2011) from where PDGFB cDNA was PCR-amplified and cloned into the retroviral expression vector pMSCV blasticidin. Wild type and K27M mutant H3.3 expression vectors were cloned into lentiviral pCDH-CMV-MCS-EF1 puro backbone and were the kind gift from Dr Peter Lewis (Lewis et al., 2013). Said expression vectors comprises DNA encoding wild type histone H3 (SEQ ID NO:1) and K27M mutant histone H3 (SEQ ID NO:3).

Cell Lines and Culture

Neural stem cells (NSCs) were isolated from the dorsal forebrain of embryonic day 12.5 (E12.5) mouse embryos. E12.5 embryos were isolated and after removal of the skin, dorsal forebrains were dissected and incubated with 0.25% trypsin-EDTA (GIBCO) at 37° C. for 20 minutes. The tissue was dissociated thoroughly with a pipette, precipitated, washed and cultured on poly-D-lysine (PDL, Sigma) and laminin (Sigma) coated plates in neural stem cell medium (50% DMEM-F12, 50% neurobasal medium, N2 and B27 supplements, sodium pyruvate, glutamax, HEPES, β-mercaptoethanol, non-essential amino acids, bovine serum albumin, heparin, 100U/ml penicillin, 100 μg/ml streptomycin, human recombinant epidermal and basic fibroblast growth factors). After 2-3 days, the expanded cells were trypsinized and frozen down in NSC medium supplemented with 10% DMSO.

Virus Production and Transduction

For production of retroviruses and lentiviruses, expression vectors were transfected into Phoenix-Eco or 293FT cells, respectively using the calcium phosphate method. After 8 hours cells were washed and cultured in desired medium. After 48 hours the medium was collected and passed through a 0.45 μm filter. For transduction, the cells were cultured in medium containing virus particles supplemented with polybrene. 48 hours after transduction, cells were harvested and cultured in selection medium.

Stereotactic Injection in Mice

All the mice experiments were approved by the Danish animal welfare authority. For stereotactic injection in Severe Combined Immunodefficient (SCID) mice (Harlan Laboratories) were performed as previously described (Caretti et al., 2011). Briefly, mice were anesthetized using isoflurane (1.5 L O₂/minute and 2.5% isoflurane) and placed in a stereotactic device (David Kopf instruments). A small incision was made to expose the skull and 0.5 mm small hole was drilled in the skull 0.8 mm below and 1 mm left to the lambda. 10,000 cells in 5 μl volume were injected 5 mm below the skull using a microsyringe (Agnthos) at a rate of 2 μl/minute. The hole in the skull was closed with bonewax (Agnthos) and the scalp was closed using clips (Agnthos). The clips were removed after one week of injection.

Protein Extraction and Immunoblotting

Cells were trypsinized, washed once with 1× phosphate buffer saline (PBS) and lysed in TOPEX+ buffer (300 mM NaCl, 50 mM Tris-HCl pH7.5, 0.5% Triton X-100, 1% SDS, 1 mM DTT, Aprotinin, Leupeptin, 0.1 mM phenylmethanesulfonyl fluoride (PMSF) and 33.33 U/mL Benzonase (EMD-Novagen)). Protein concentrations in the cell lysates were measured by Bradford reagent (Bio-Rad). Cell lysates were separated by SDS-PAGE and transferred to nitrocellulose membrane. The antibodies used for the immunoblotting were antibodies specifically recognising H3K27me3 (C36B11, Cell Signaling), H3K27me2, p16^{Ink4a}, p53, H3K27M, actin, H3 and Ezh2.

Chromatin Immunoprecipitation (ChIP)

Cells were cross-linked with 1% formaldehyde for 10 minutes at room temperature. Glycine was added at a final concentration of 125 mM to quench the formaldehyde. Cells were then washed twice with PBS and harvested in SDS buffer (50 mM Tris at pH 8.1, 0.5% SDS, 100 mM NaCl, 5 mM EDTA). Cells were pelleted, resuspended in Triton-X IP buffer (100 mM Tris at pH 8.6, 0.3% SDS, 1.7% Triton X-100, and 5 mM EDTA) and the chromatin was sonicated to obtain DNA fragments of <1000 bp with average DNA fragment size of 300 bp.

100 μg chromatin was pre-cleared with protein A Sepharose beads (GE healthcare) for 1-2 hours and incubated with the indicated antibody overnight at 4° C. Next day, protein A Sepharose beads were added and incubated for 3 hours at 4° C. Beads were washed three times with low salt buffer (1% Triton X-100, 0.1% SDS, 150 mM NaCl, 2 mM EDTA, pH 8.0, 20 mM Tris-HCl, ph 8.0) and once with high salt buffer (1% Triton X-100, 0.1% SDS, 500 mM NaCl, 2 mM EDTA, 20 mM Tris-HCl, pH 8.0). Beads were incubated with elution buffer (1% SDS, 0.1M sodium bicarbonate) at 65° C. for 4 hours to overnight to elute DNA and associated proteins. The DNA was isolated and purified using QIAquick PCR purification kit (Qiagen) and eluted in 100 μl elution buffer. The ChIP DNA was diluted 10 times in water and subjected to qPCR analysis using 1× SYBR green master mix (Roche Applied Science) and LightCycler 480 instrument (Roche Applied Science). The primers used for the analysis are listed in Table 2.

Cell Proliferation Assay

100,000 neural stem cells prepared as described above and transduced with virus containing DNA encoding wild type histone H3 (SEQ ID NO:1) or K27M mutant histone H3 (SEQ ID NO:3) were plated in duplicate in six well plates and treated with either DMSO or with 3 μM of an inhibitor of EZH2. The inhibitor was either GSK343, which is the compound of formula B or EPZ6438, which is the compound of formula C. Cells were harvested and counted using Neubauer chamber every 3-4 days.

Colony Formation Assay

For colony formation assay, 2000 cells were plated on PDL and laminin coated 6-well plates in duplicates and treated with DMSO, GSK343 or EPZ6438. Colonies formed after 9 days were fixed and stained with crystal violet.

### Example 2

Assay for Determining Whether a Compound is an Inhibitor of EZH2

Compounds can be evaluated for their ability to inhibit the methyltransferase activity of EZH2 within the PRC2 complex using the following assay. Human PRC2 complex is prepared by co-expressing each of the 5 member proteins (EZH2, EED, SUZ12, RbAp48, AEBP2) in Sf9 cells followed by co-purification. The proteins may be expressed as tagged versions, e.g. EZH2 may be expressed as FLAG-EZH2. The tag can be used for purification.

The sequences of the proteins of the human PRC2 complex are available to the skilled person. For example useful sequences are available under the following Genebank accession numbers:

| | |
|---|---|
| EZH2 | Sequence provided as SEQ ID NO: 4 herein |
| EED | NP_003788.2 GI:24041020 |
| SUZ12 | NP_056170.2 GI:197333809 |
| RbAp48 (also | NP_005601.1 GI:5032 |
| known as RBBP4) | |
| AEBP2 | NP_694939.2 GI:166795262 |

Enzyme activity is measured in a scintillation proximity assay (SPA) where a tritiated methyl group is transferred from 3H-SAM to a lysine residue on Histone H3 of a mononucleosome, purified from HeLa cells. Mononucleosomes are captured on SPA beads and the resulting signal is read on a ViewLux plate reader. SPA beads are e.g. available from Perkin Elmer, United States. This may be done as described in Garapaty-Rao et al, 2013 Identification of EZH2 and EZH1 small molecule inhibitors with selective impact on diffuse large B cell lymphoma cell growth. Chemistry and Biology 20, 1329-1339.

Part A Compound Preparation

1. Prepare 10 mM stock of compounds e.g. from solid in 100% DMSO.

2. Set up an 11-point serial dilution (1:3 dilution, top concentration 10 mM) in 100% DMSO for each test compound in a 384 well plate leaving columns 6 and 18 for DMSO controls.

3. Dispense 100 nL of compound from the dilution plate into reaction plates (Grenier Bio-One, 384-well, Cat#784075).

Part B Reagent Preparation

Prepare the following solutions:

1. 50 mM Tris-HCl, pH 8: Per 1 L of base buffer, combine 1 M Tris-HCl, pH 8 (50 mL) and distilled water (950 mL).

2. lx Assay Buffer: Per 10 mL of lx Assay Buffer, combine 50 mM Tris-HCl, pH 8 (9958 uL), 1 M MgCl2 (20 uL), 2 M DTT (20 uL), and 10% Tween-20 (2 uL) to provide a final concentration of 50 mM Tris-HCl, pH 8, 2 mM MgCl2, 4 mM DTT, 0.002% Tween-20.

3. 2× Enzyme Solution: Per 10 mL of 2× Enzyme Solution, combine lx Assay Buffer and PRC2 complex to provide a final enzyme concentration of 10 nM.

4. SPA Bead Suspension: Per 1 mL of SPA Bead Suspension, combine PS-PEI coated LEAD Seeker beads (40 mg) and ddH20 (1 mL) to provide a final concentration of 40 mg/mL.

5. 2× Substrate Solution: Per 10 mL of 2× Substrate Solution, combine lx Assay Buffer (9728.55 uL), 800 ug/mL mononucleosomes (125 uL), 1 mM cold SAM (4 uL), and 7.02 uM 3H-SAM (142.45 uL; 0.55 mCi/mL) to provide a final concentration of 5 ug/mL nucleosomes, 0.2 uM cold SAM, and 0.05 uM 3H-SAM.

6. 2.67× Quench/Bead Mixture: Per 10 mL of 2.67× Quench/Bead Mixture, combine dd3/40 (9358 uL), 10 mM cold SAM (267 uL), 40 mg/mL Bead Suspension (375 uL) to provide a final concentration of 100 uM cold SAM and 0.5 mg/mL SPA beads.

Part C. Assay Reaction in 384-well Grenier Bio-One Plates

Compound Addition

1. Dispense 100 nL/well of lOOx Compound to test wells (as noted above).

2. Dispense 100 nL/well of 100% DMSO to columns 6 & 18 for high and low controls, respectively.

Assay

1. Dispense 5 uL/well of 1× Assay Buffer to column 18 (low control reactions).

2. Dispense 5 uL/well of 2× Enzyme Solution to columns 1-17, 19-24.

3. Spin assay plates for ˜1 minute at 500 rpm.

4. Stack the assay plates, covering the top plate.

5. Incubate the compound/DMSO with the enzyme for 30 minutes at room temperature.

6. Dispense 5 uL/well of 2× Substrate Solution to columns 1-24.

7. Spin assay plates for ˜1 minute at 500 rpm.

8. Stack the assay plates, covering the top plate.

9. Incubate the assay plates at room temperature for 1 hour.

Quench/Bead Addition

1. Dispense 5 uL/well of the 3× Quench/Bead Mixture to columns 1-24.

2. Seal the top of each assay plate with adhesive TopSeal.

3. Spin assay plates for ˜1 minute at 500 rpm.

4. Equilibrate the plates for >20 min.

Read Plates

1. Read the assay plates on the Viewlux Plate Reader utilizing the 613 nm emission filter with a 300 s read time.

Reagent addition can be done manually or with automated liquid handler.
The final DMSO concentration in this assay is 1%.
The positive control is in column 6; negative control is in column 18.
Final starting concentration of compounds is 100 μM.

Part D. Data Analysis

Percent inhibition is calculated relative to the DMSO control for each compound concentration and the resulting values are fit using standard IC50 fitting parameters within the ABASE data fitting software package.

Compounds having an IC50<10 μM may be considered as inhibitors of EZH2. For example compounds having an IC50 value in the range from about 1 nM to about 10 μM may be considered inhibitors of EZH2. More potent inhibitors of EZH2 may have an IC50<500 nM, such as in the range from about 1 nM to about 500 nM. Very potent inhibitors of EZH2 have an IC50<50 nM.

The compound of formula B provided above has an IC50 of 5 nM when determined as described in this example.

### Example 3

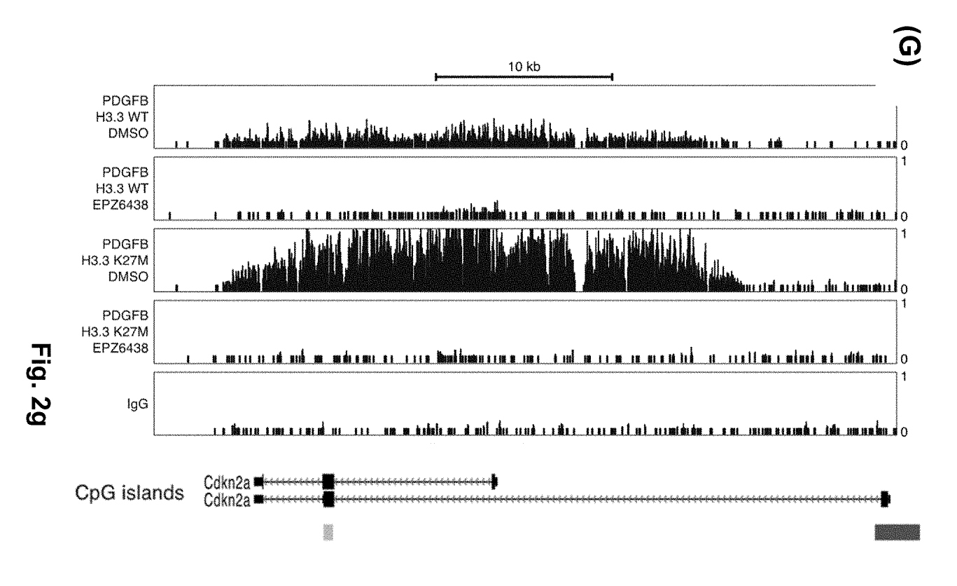

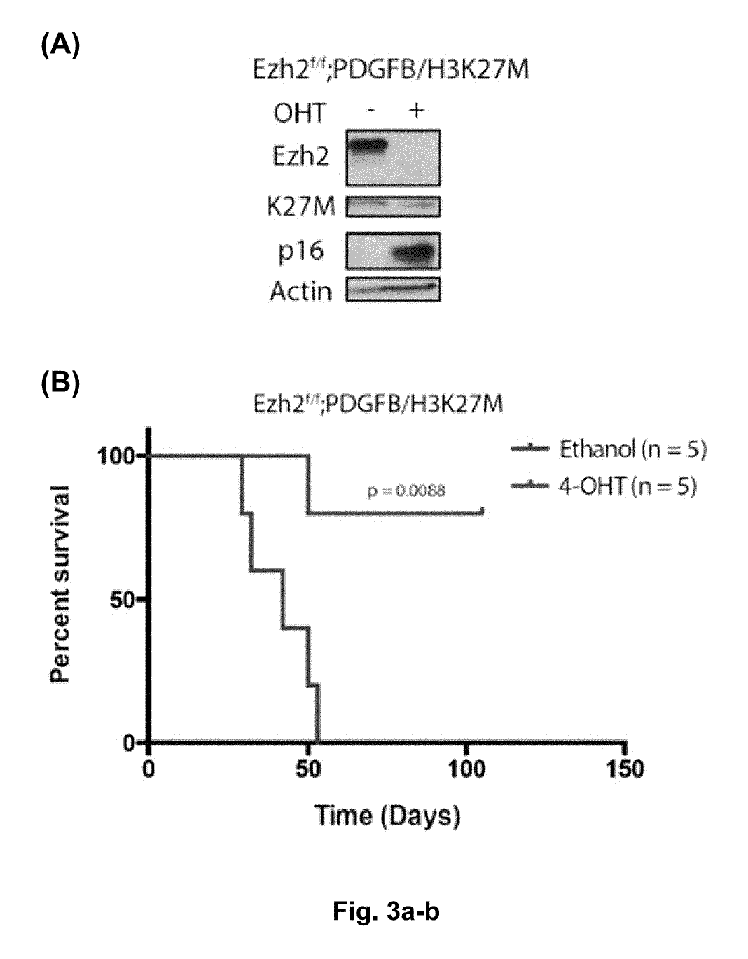

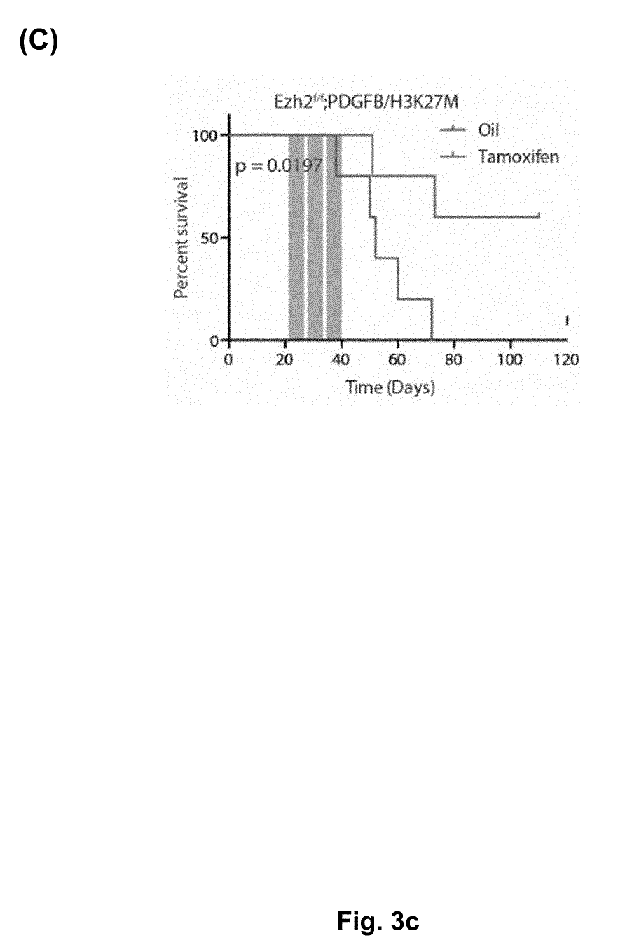

PDGFB/H3K27M NSCs in mice with Ezh2^{f/f}; CreER (Ezh2^{f/f};PDGFB/H3K27M) background were generated. In these mice Ezh2 could be conditionally deleted. Treatment with 4-hydroxytamoxifen(4-OHT) resulted in loss of Ezh2 expression (see FIG. 3a), and when injected into the mouse pons, the mice with the 4-OHT-treated cells showed significantly longer survival than the mice injected with control ethanol-treated cells (see FIG. 3b). To delete Ezh2 in tumour cells in vivo, we injected Ezh2f/f; PDGFB/H3K27M NSCs in the mouse pons, and after 3 weeks we treated the mice with tamoxifen. Tamoxifen-treated mice showed significantly longer survival than control oil-treated mice (FIG. 3c), indicating that Ezh2 is also essential for in vivo tumour cell growth.

Cell culture and injection of mice were performed essentially as described in Example 1.: NSCs (Ezh2f/f; CreER) were prepared as described in Example 1, and transduced with viruses expressing PDGFB and H3.3 WT (SEQ ID NO:1) or H3K27M (SEQ ID NO:3). The cell culture and injection of mice were performed essentially as described in Example 1.

### REFERENCES

V. Caretti et al., Monitoring of tumor growth and post-irradiation recurrence in a diffuse intrinsic pontine glioma mouse model. Brain Pathol. 21, 441-451 (2011)
Shivani Garapaty-Rao et al., Identification of EZH2 and EZH1 Small Molecule Inhibitors with Selective Impact on Diffuse Large B Cell Lymphoma Cell Growth. Chemistry & Biology 20, 1329-1339, (2013)
Y. Jiang, M. Boije, B. Westermark, L. Uhrbom, PDGF-B Can sustain self-renewal and tumorigenicity of experimental glioma-derived cancer-initiating cells by preventing oligodendrocyte differentiation. Neoplasia 13, 492-503 (2011).
S. K. Knutson et al., A selective inhibitor of EZH2 blocks H3K27 methylation and kills mutant lymphoma cells. Nat Chem Biol 8, 890-896 (2012).
S. K. Knutson et al., Selective Inhibition of EZH2 by EPZ-6438 Leads to Potent Antitumor Activity in EZH2-Mutant Non-Hodgkin Lymphoma. Mol. Cancer Ther. (2014), doi:10.1158/1535-7163.MCT-13-0773.
P. W. Lewis et al., Inhibition of PRC2 activity by a gain-of-function H3 mutation found in pediatric glioblastoma. Science 340, 857-861 (2013).
M. T. McCabe et al., EZH2 inhibition as a therapeutic strategy for lymphoma with EZH2-activating mutations. Nature 492, 108-112 (2012).
Wei Qi et al., Selective inhibition of Ezh2 by a small molecule inhibitor blocks tumor cells proliferation. PNAS, vol. 109, no. 52, p. 21360-21365 (2012)

1. An inhibitor of EZH2 for use in the treatment of cancer in an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

2. The inhibitor according to claim 1, wherein the inhibitor is a compound capable of reducing or completely inhibiting trimethylation of K27 of histone H3 by EZH2.

3. The inhibitor according to claim 1, wherein the inhibitor is a compound capable of reducing or completely inhibiting trimethylation of K27 of histone H3 by PRC2.

4. The inhibitor according to claim 1, wherein the inhibitor has an IC₅₀ of <10 μM, more preferably <500 nM, even more preferably <50 nM with regard to inhibiting trimethylation of K27 of histone H3 by EZH2.

5. The inhibitor according to claim 1, wherein the inhibitor has an IC₅₀ of <10 μM, more preferably <500 nM, even more preferably <50 nM with regard to inhibiting trimethylation of K27 of histone H3 by PRC2.

6. The inhibitor according to claim 1, wherein the inhibitor contains the core structure:

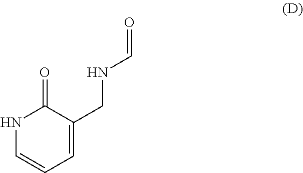 [SMILES: O=CNCc1ccc[nH]c1=O]

7. The inhibitor according to claim 1, wherein the inhibitor is a compound of formula B 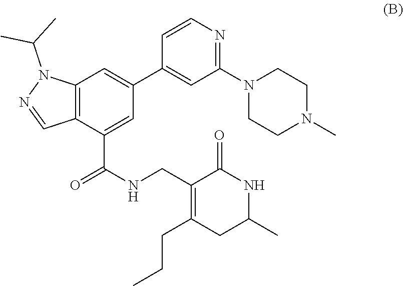 [SMILES: CCCC1=C(CNC(=O)c2cc(-c3ccnc(N4CCN(C)CC4)c3)cc3c2cnn3C(C)C)C(=O)NC(C)C1]
or a solvate or a pharmaceutically acceptable salt thereof.

8. The inhibitor according to claim 1, wherein the inhibitor is a compound of formula C 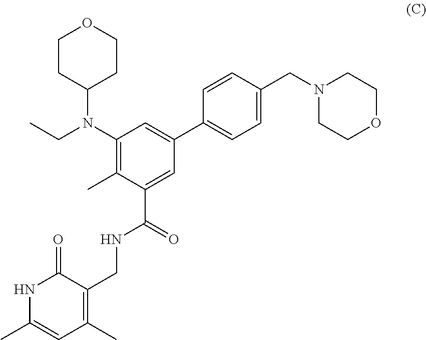 [SMILES: CCN(c1cc(-c2ccc(CN3CCOCC3)cc2)cc(C(=O)NCc2c(C)cc(C)[nH]c2=O)c1C)C1CCOCC1]
or a solvate or a pharmaceutically acceptable salt thereof.

9. The inhibitor according to claim 1, wherein the cancer is a cancer characterised by expression of mutated histone H3 having a mutation of amino acid number 27 from lysine to methionine or isoleucine and/or the cancer is characterised by a mutation in at least one gene encoding histone H3, wherein the mutated histone H3 gene encodes mutated histone H3 having a mutation of amino acid number 27 from lysine to methionine or isoleucine.

10. The inhibitor according to claim 1, wherein the cancer is a cancer characterised by expression of mutated histone H3 having a mutation of amino acid number 27 from lysine to methionine and/or the cancer is characterised by a mutation in at least one gene encoding histone H3, wherein the mutated histone H3 gene encodes mutated histone H3 having a mutation of amino acid number 27 from lysine to methionine.

11. The inhibitor according to claim 1, wherein histone H3 is histone H3.3 of SEQ ID NO:1.

12. The inhibitor according to claim 1, wherein histone H3 is histone H3.1 of SEQ ID NO:2.

13. The inhibitor according to claim 1, wherein the cancer further is characterised by the presence of a gene encoding p16^{Ink4a}.

14. The inhibitor according to claim 13, wherein p16^{INK4A} is p16^{INK4A} of SEQ ID NO:5.

15. The inhibitor according to claim 1, wherein the cancer is a diffuse intrinsic pontine glioma.

16. A method for predicting the efficacy of treatment of a cancer with an inhibitor of EZH2 in an individual in need thereof, said method comprising the steps of
i) providing a sample comprising cells of said cancer from said individual,
ii) determining whether said cells contain a gene encoding p16^{INK4A},
wherein the presence of a gene encoding p16^{INK4A} in said cells is indicative of efficacy of treatment of the cancer in said individual with an inhibitor of EZH2.

17. An inhibitor of EZH2 for use in the treatment of cancer in an individual in need thereof, wherein said cancer is a cancer characterised by containing a gene encoding p16^{INK4A}.

18. The method or the inhibitor according to claim 16, wherein said cancer is a cancer characterised by expression of mutated histone H3 having a mutation of amino acid number 27 and/or a cancer characterised by a mutation in at least one gene encoding histone H3, wherein the mutated histone H3 gene encodes mutated histone H3 having a mutation of amino acid number 27.

19. The method or the inhibitor according to claim 16, wherein the cancer is a cancer characterised by expression of mutated histone H3 having a mutation of amino acid number 27 from lysine to methionine or isoleucine and/or the cancer is characterised by a mutation in at least one gene encoding histone H3, wherein the mutated histone H3 gene encodes mutated histone H3 having a mutation of amino acid number 27 from lysine to methionine or isoleucine.

20. The method or the inhibitor according to claim 16, wherein p16^{INK4A} is p16^{INK4A} of SEQ ID NO:5.

21. A method of treatment of cancer comprising administering a therapeutically effective amount of an inhibitor of EZH2 to an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

22. The method according to claim 21, wherein the cancer is a cancer characterised by expression of mutated histone H3 having a mutation of amino acid number 27 from lysine to methionine or isoleucine and/or the cancer is characterised by a mutation in at least one gene encoding histone H3, wherein the mutated histone H3 gene encodes mutated histone H3 having a mutation of amino acid number 27 from lysine to methionine or isoleucine.

23. A method for treatment of cancer in an individual in need thereof, wherein the method comprises the steps of:
i) Obtaining information of whether cells of the cancer from said individual comprises a gene encoding p16^{INK4A}; and
ii) if said cancer cells contain a gene encoding p16^{INK4A}, then administering a therapeutically effective amount of said inhibitor of EZH2 to said individual
thereby treating cancer in said individual.

24. The method according to claim 23, wherein the cancer is a cancer characterised by expression of mutated histone H3 having a mutation of amino acid number 27 from lysine to methionine or isoleucine and/or the cancer is characterised by a mutation in at least one gene encoding histone H3, wherein the mutated histone H3 gene encodes mutated histone H3 having a mutation of amino acid number 27 from lysine to methionine or isoleucine.

25. The method according to claim 23, wherein p16^{Ink4a} is p16^{Ink4a} of SEQ ID NO:5.

26. The method according to claim 16, wherein the inhibitor of EZH2 is a compound capable of reducing or completely inhibiting trimethylation of K27 of histone H3 by EZH2.

27. Use of an inhibitor of EZH2 for the preparation of a medicament for treatment of a cancer in an individual in need thereof, wherein said cancer is a cancer characterised by expression of mutated histone H3 having a mutation of amino acid number 27 and/or the cancer is characterised by a mutation in at least one gene encoding histone H3, wherein the mutated histone H3 gene encodes mutated histone H3 having a mutation of amino acid number 27.

28. Use according to claim 27, wherein the inhibitor of EZH2 is a compound capable of reducing or completely inhibiting trimethylation of K27 of histone H3 by EZH2.

29. Use according to claim 27, wherein the cancer is a cancer characterised by expression of mutated histone H3 having a mutation of amino acid number 27 from lysine to methionine or isoleucine and/or the cancer is characterised by a mutation in at least one gene encoding histone H3, wherein the mutated histone H3 gene encodes mutated histone H3 having a mutation of amino acid number 27 from lysine to methionine or isoleucine.
